(19) United States
(12) Patent Application Publication
(10) Pub. No.: US 2025/0187919 A1
(43) Pub. Date: Jun. 12, 2025

(54) HELIUM RECOVERY FROM THE NATURAL GAS IN PETROCHEMICAL PLANTS

(71) Applicant: Seyed Ahmad Banyhashemian, Tehran (IR)

(72) Inventor: Seyed Ahmad Banyhashemian, Tehran (IR)

(21) Appl. No.: 19/052,402

(22) Filed: Feb. 13, 2025

(51) Int. Cl. C01B 23/00; B01D 53/00; B01D 53/02; B01D 53/14; B01D 53/22; C01B 5/00; C01B 32/40; C01B 32/50; C07C 1/12; C07C 29/151

(52) U.S. Cl. CPC C01B 23/0089; B01D 53/002; B01D 53/02; B01D 53/1493; B01D 53/228; B01D 53/229; C01B 5/00; C01B 23/0047; C01B 23/0063; C01B 32/40; C01B 32/50; C07C 1/12; C07C 29/1516; B01D 2252/103; B01D 2253/102; B01D 2256/18; B01D 2256/22; B01D 2257/102; B01D 2257/108; B01D 2257/11; B01D 2257/502; B01D 2257/504; B01D 2257/7025; B01D 2257/80; B01D 2259/402; C01B 2210/0012; C01B 2210/0017; C01B 2210/0031; C01B 2210/0046; C01B 2210/005; C01B 2210/0051; C01B 2210/0053; C01B 2210/0062; C01B 2210/007; C01P 2006/80

(57) ABSTRACT

The present invention provides systems and methods for simultaneously producing a high-purity helium gas product, a methanol-water liquid mixture, and a methane-rich fuel product from a hydrogen-rich feedstock gas containing helium by treating the hydrogen-rich feedstock gas containing helium and carbon dioxide in a reverse water gas shift unit (1500) to produce carbon monoxide, which is then treated in a methanol production unit (300) and a methanol absorption unit (400) to produce a methanol-aqueous solution and a methanol-free gas. The methanol-free gas is then treated in a methane production unit (500) to produce methane, which is then treated in a carbon dioxide recovery membrane unit (1100) and a cryogenic nitrogen rejection unit (600) to produce the methanol-water liquid mixture, the methane-rich fuel product, and a helium-rich gas. The helium-rich gas is then treated to produce the high-purity helium gas product.

### BACKGROUND OF THE INVENTION

Helium recovery from the natural gas is a promising way for increasing the economic value of petrochemical plants. A variety of helium recovery technologies are available, including membrane separation, cryogenic separation, and pressure swing adsorption separation. The best technology for a particular application would depend on the natural gas composition and the desired purity of a helium product. helium recovery technologies faces certain challenges. One is the high cost of cooling gases to cryogenic temperatures. Another challenge is the conversion of most of the valuable hydrogen feedstock into water or fuel as a low-value products.

The conventional reverse water gas shift (RWGS) process converts carbon dioxide into carbon monoxide using hydrogen. However, methanation reactions occur simultaneously, consuming hydrogen and carbon dioxide to produce methane, which is an undesired byproduct. This competition is a major challenge in RWGS and is exacerbated by higher pressure conditions, which favor methane production according to Le Chatelier's principle. Therefore, there is a need for a method to suppress methane formation and improve carbon monoxide yield in RWGS processes.

Methanol synthesis from carbon monoxide and hydrogen faces the challenge of methanation as a competitor reaction. Also other existing challenge in traditional methanol production with series-connected methanol reactors is the presence of methanol and water vapor in the gases entering downstream methanol reactors. These components can hinder further methanol production in subsequent methanol reactors.

Helium recovery from a high content nitrogen and carbon monoxide gas by a cryogenic process face the challenge of very low cryogenic temperature requirements and special rotating equipment such as oil free compressors and turboexpanders.

### FIELD OF THE INVENTION

The present invention provides systems and methods for obtaining a high-purity helium gas product and simultaneously producing a methanol-water liquid mixture and a methane-rich fuel product from a hydrogen-rich feedstock gas containing helium, a carbon dioxide-rich feedstock gas, and an inert methane-rich gas. The synthetic loop purge gas as a hydrogen-rich feedstock gas containing helium is produced by petrochemical plants that use the natural gas as a feedstock, such as methanol, dimethyl ether (DME), or heavy hydrocarbons (Fischer-Rosch process) plants. The present invention provides systems and methods to limit the amount of gases processed in cryogenic temperatures and the amount of hydrogen converted to water and fuel. The present invention provides systems and methods to tackle the challenge of methane formation in RWGS processes by introducing an inert methane-rich gas stream to the RWGS reaction feed mixture at a low pressure. The present invention provides systems and methods for methanol synthesis within series-connected methanol reactors comprising introducing a controlled amount of the inert methane-rich gas into the primary feedstock and using solvent absorber columns with a high-boiling inert solvent to remove methanol and water vapor in the gases entering downstream methanol reactors. The present invention provides systems and methods to remove nitrogen, carbon monoxide, and a main bulk of methane gases from a helium-rich gas by methane liquification in coldbox and absorption by recycled high-purity liquid methane stream in a methane absorber tower. After that, hydrogen and remain methane are removed from the helium-rich gas by oxygen in a hydrogen catalytic combustion reactor.

### BACKGROUND ART

Helium is a valuable resource that is used in a variety of industries and applications, such as aerospace, electronics, food processing, MRI machines, cryogenics, and welding. A small amount of helium is present in most natural gas fields. In petrochemical plants that use the natural gas as a feedstock, methane, the main component of the natural gas, is transformed into carbon monoxide and hydrogen at a high temperature in a steam reforming process and an autothermal reforming process by steam and oxygen. A mixture of carbon monoxide and hydrogen is known as syngas.

Within a synthesis gas loop, the syngas undergoes multiple passes over a catalyst bed, facilitating the production of various substances, including methanol, dimethyl ether (DME), or heavy hydrocarbons (Fischer-trophy process) depending on the type of catalyst. Helium of the natural gas and other inert gases, such as nitrogen, accumulate in the synthesis loop. In order to continue the process, inert gases must be discharged from the synthesis loop, so a portion of the circulating synthesis gas is discharged from the synthesis loop that is called synthetic loop purge gas. The synthetic loop purge gas is a hydrogen-rich gas containing helium and other gaseous components.

A variety of helium recovery technologies are available, including membrane separation, cryogenic separation, and pressure swing adsorption separation. The best technology for a particular application would depend on the synthetic loop purge gas composition and the desired purity of a helium product.

The concentration of helium in the synthetic loop purge gas is much higher than the concentration of helium in the petrochemical plants natural gas feedstock. Helium recovery from the synthetic loop purge gas is a promising technology for increasing thee economic value of petrochemical plants. First, helium is a high-value commodity that can be sold to generate additional revenue. Second, helium recovery with consumption of carbon dioxide feedstock can reduce the environmental impact of petrochemical plants by reducing the emission of greenhouse gases.

The synthetic loop purge gas is rich in hydrogen and is typically used as a fuel to generate heat in main (primary) reformers. For example, the synthetic loop purge gas provides more than 50.0% of a fuel gas energy in methanol plants main reformers. Converting the synthetic loop purge gas to other fuel does not save energy. Instead, modern methanol plants send the synthetic loop purge gas to a hydrogen recovery unit, where hydrogen is recovered. Then, hydrogen combined with a carbon dioxide feedstock to be sent back to the methanol plants. This configuration increases methanol production, but it also increases net fuel gas consumption, net oxygen consumption, and operational expenditures (OPEX) of the methanol plants.

The present invention provides systems and methods for obtaining a high-purity helium gas product from the hydrogen-rich feedstock gas containing helium (like synthetic loop purge gas) while maximizing a methanol-water liquid mixture production and minimizing a methane-rich fuel production. This arrangement maximizes the production of valuable products (helium and methanol) instead of using the hydrogen-rich feedstock gas containing helium as a fuel gas, which provides an economic incentive to eliminate the use of the hydrogen-rich feedstock gas containing helium as a fuel gas. Also the present invention provides systems and methods for carbon dioxide consumption as a feedstock.

The present invention provides systems and methods to mitigate methane formation during the reverse water gas shift (RWGS) process. This is achieved by introducing a controlled flow of an inert methane-rich gas stream, to the RWGS reaction mixture at a low pressure.

The present invention proposes a configuration for methanol production utilizing series-connected methanol reactors, characterized by the introduction of a controlled amount of an inert methane-rich gas into a main syngas feedstock, followed by the utilization of solvent absorber columns containing a high-boiling inert solvent to capture methanol and water vapor in gases entering downstream methanol reactors.

The present invention provides systems and methods to remove nitrogen, carbon monoxide, and a main bulk of methane gases from a helium-rich gas by methane liquification in coldbox and absorption by recycled high-purity liquid methane stream in a methane absorber tower.

J.P. Pat. No. 2022012436A discloses a process for methanol and methane co-production from the syngas without helium recovery, while the present invention provides systems and methods for helium recovery and simultaneous production of methane and methanol.

J.P. Pat. No. 2022012436A discloses a process for carbon monoxide production from carbon dioxide and hydrogen by reverse water gas shift (RWGS) reaction. During the conversion of carbon dioxide and hydrogen using the RWGS process, the methanation reactions act as unwanted competitors. The choice between forming carbon monoxide or methane during the conversion of carbon dioxide and hydrogen is highly influenced by pressure. Higher pressure favors methane production as it involves fewer gas molecules, following Le Chatelier's principle. The prior art disclosed in J.P. Patent No. 2022012436A fails to provide a concrete solution to the identified problem. In contrast, the present invention provides systems and methods to reduce methane production in RWGS reactors. The present invention proposes mixing an inert methane-rich gas with RWGS reactors main feed and also simultaneously lowering a reaction pressure.

J.P. Pat. No. 2022012436A discloses a process for methanol production that involves recirculating unreacted feed contents and separating unwanted substances. However, this recirculation system is only proposed for use when the methanol and methane production sections operate in parallel. In contrast, the present invention provides systems and methods for operating the methanol and methane production sections in series-connection. This approach utilizes unreacted carbon dioxide recirculation and separation of other substances.

J.P. Pat. No. 2022012436A discloses a single methanol reactor for methanol production. In contrast to the prior art, the present invention provides systems and methods for obtaining methanol in series-connected methanol reactors. To reduce methanol and water vapor in gases entering downstream methanol reactors and prevent them from hindering methanol production, the present invention proposes using solvent absorber columns between all of the methanol reactors. These columns use a high-boiling inert solvent like tetraethylene glycol dimethyl ether (tetraglyme) (TEGDE) that selectively absorbs methanol and water. Also during the conversion of carbon monoxide and hydrogen using the methanol reaction, the methanation reactions act as unwanted competitors. The prior art disclosed in J.P. Patent No. 2022012436A fails to provide a concrete solution to the identified problem. In contrast, the present invention provides systems and methods to reduce methane production in methanol reactors. The present invention proposes mixing an inert methane-rich gas with methanol reactors main feed. These configuration maximizes hydrogen conversion to methanol and is the most economical choice, as it reduces the amount of remaining hydrogen that can be converted to methane.

J.P. Pat. No. 2022012436A discloses a method for reducing the outlet temperature of methanation reactors by recirculating more than 60.0% (mole) of the product from the methanation section. In contrast, the present invention provides systems and methods for producing methane by recycling unreacted carbon dioxide and separating other substances. Recycling carbon dioxide instead of other substances reduces the amount of cooling fluid required in methanation reactors due to the higher molar specific heat capacity of carbon dioxide than many other gases, which in turn reduces the energy consumption of recycling compressors.

J.P. Pat. No. 2022012436A discloses a method for reducing the outlet temperature of methanation reactors by recirculating more than 60.0% (mole) of the product from the methanation section. This configuration leads to the accumulation of inert gases in the loop and decreases the methanation reactions when nitrogen or other inert gases are present in feedstock. This configuration requires more catalyst in methanation reactors. In contrast, the present invention, has a low accumulation of inert gases in all of the loops. Most feedstocks in petrochemical plants contain inert gases, and the cost of eliminating inert gases from a feedstock is very high.

U.S. Pat. No. 5,771,714 discloses a cooling process to recover helium from the natural gas using refrigeration cycles. This method can be used in natural gas liquefaction units to recover helium, but the energy cost of helium recovery is high due to amount of methane that to be liquified.

In U.S. Pat. No. 5,632,803, helium gas is separated by membranes and two pressure swing adsorption units. This method is not economical for feedstocks containing high amounts of hydrogen because without hydrogen converting this method needs a lot of membrane modules for separating hydrogen from helium.

U.S. Pat. No. 3,807,185 discloses a multi-stage cooling and liquefaction process for recovering helium from vent gas streams generated by ammonia plants. Due to the inherently low helium concentration in the main feed stream, this method suffers from high energy consumption during cooling and liquefaction, resulting in increased cost for helium recovery.

In U.S. Pat. No. 8,152,898B2, the metal hydrides are used to separate helium and hydrogen. The metal hydrides are materials that can adsorb hydrogen more selectively than helium and other gases. The challenge of this method is the ratio of the adsorbed hydrogen weight to the adsorbent weight. For example, in a methanol unit, up to 1500.0 kg/hour of hydrogen is purged, which needs to be captured by the metal hydrides. In current metal hydride technologies, the ratio of hydrogen to metal hydride is less than 0.09 kg/kg, so a very large amount of metal hydride would be needed to commercialize this method.

C.N. Pat. No. 108394878B employs the conversion of carbon dioxide to methane for simultaneous hydrogen removal and production of a helium-enriched stream. However, complete conversion of hydrogen to methane offers limited economic benefit. Alternatively, converting hydrogen to methanol presents a more favorable economic proposition due to its inherent value.

C.N. Pat. No. 108394878B uses helium, nitrogen and methane as inert gases in methanation reactions at the inlet of methanation reactors to decrease the outlet temperature of methanation reactors. In contrast, the present invention provides systems and methods for producing methane by recycling unreacted carbon dioxide and separating other substances. Recycling carbon dioxide instead of other substances reduces the amount of cooling fluid required in methanation reactors due to the higher molar specific heat capacity of carbon dioxide than many gases, which in turn reduces the energy consumption of recycling compressors. The prior art disclosed in C.N. Pat. No. 108394878B fails to provide a concrete solution to the high temperature problem of methanation reactors outlet while feedstock gas contains low concentration of helium and high concentration of nitrogen and methane as inert gases.

C.N. Pat. No. 113460982A uses the conversion of carbon monoxide to methane to remove hydrogen and produce a helium-rich stream. To control the methanation reaction temperature, it is proposed to inject hydrogen-free carbon monoxide gradually. From an economic point of view, carbon monoxide production without hydrogen is an expensive process that uses a refrigeration cycle and increases the cost of helium production. Additionally, converting most of the valuable hydrogen to methane does not create additional economic value, while converting hydrogen to methanol has a higher economic value.

In C.N. Pat. No. 211612197U, uses the conversion of carbon monoxide and carbon dioxide to methane for removing hydrogen and produce a helium-rich stream. However, converting most of the valuable hydrogen to methane does not create additional economic value. Converting hydrogen to methanol has a higher economic value. C.N. Pat. No. 211612197U does not propose any solution to decrease the outlet temperature of methanation reactors. In contrast, the present invention provides systems and methods for producing methane by recycling unreacted carbon dioxide and separating other substances. Recycling carbon dioxide instead of other substances reduces the amount of cooling fluid required in methanation reactors due to the higher molar specific heat capacity of carbon dioxide than many gases, which in turn reduces the energy consumption of recycling compressors.

In C.N. Pat. No. 1880414A, a reverse water gas shift reaction is described to convert carbon dioxide to carbon monoxide in order to improve the hydrogen to carbon monoxide ratio in a circulating synthesis gas in a methanol production loop. C.N. Pat. No. 1880414A does not claim to produce helium. It discloses a process for carbon monoxide production from carbon dioxide by reverse water gas shift (RWGS) reaction. During the conversion of carbon dioxide and hydrogen using a RWGS process, methanation reactions act as unwanted competitors. The choice between forming carbon monoxide or methane during the conversion of carbon dioxide and hydrogen is highly influenced by pressure. Higher pressure favors methane production as it involves fewer gas molecules, following Le Chatelier's principle. The prior art disclosed in C.N. Pat. No. 1880414 A fails to provide a concrete solution to the identified problem. In contrast, to reduce methane production in RWGS reactors, the present invention proposes mixing a methane gas with the main feeds and also simultaneously lowering the RWGS reaction pressure.

In C.N. Pat. No. 1880414A, the output of the reverse water gas shift (RWGS) reactor is sent to the methanol synthesis loop. A disadvantage of this configuration is the increased accumulation of inert gases, such as nitrogen and methane, within the methanol production synthesis loop. This accumulation reduces the performance of the methanol catalyst. In contrast, the present invention provides systems and methods for methanol synthesis within series-connected methanol reactors, unreacted carbon dioxide recirculation and using solvent absorber columns with a high-boiling inert solvent to remove methanol and water vapor in gases entering downstream methanol reactors.

R.U. Pat. No. 2478569C1 proposes a process for recovering helium from a synthetic loop purge gas generated during methanol production. The process involves extracting hydrogen and helium via adsorption on activated carbon or zeolite, followed by catalytic oxidation to remove hydrogen and subsequent helium capture using a membrane or pressure swing adsorption (PSA) units. The synthetic loop purge gas is primarily utilized as fuel for the methanol unit's main reformers, contributing over 50.0% of their fuel energy source. Converting this energy into steam increases the unit's fuel gas consumption, leading to higher operational expenditures (OPEX). In contrast, the present invention proposes converting hydrogen to methanol that presents a more favorable economic proposition due to its inherent value.

### SUMMARY OF THE INVENTION

The present invention provides systems and methods for obtaining a high-purity helium gas product from a hydrogen-rich feedstock gas containing helium (like synthetic loop purge gas), while simultaneously maximizing a methanol-water liquid mixture production and minimizing a methane-rich fuel production. The hydrogen-rich feedstock gas containing helium (like synthetic loop purge gas) is produced by petrochemical plants that use the natural gas as a feedstock

Within a petrochemical plant, the synthetic loop purge gas, characterized by its high hydrogen content, is traditionally employed as a fuel source to generate heat for main reformers. For example, over 50.0% of a methanol plant main reformer fuel energy originates from the synthetic loop purge gas. However, it is important to recognize that converting the synthetic loop purge gas to alternative fuels does not yield any energy conservation benefits.

In contemporary methanol plants, a more sophisticated approach is implemented. The synthetic loop purge gas is directed to a dedicated hydrogen recovery unit (HRU), where hydrogen is efficiently extracted and subsequently combined with carbon dioxide-rich feedstock gas. Then, this enriched mixture is reintroduced into the methanol plant. While this configuration undoubtedly leads to an increase in methanol production, it simultaneously entails an augmentation in net fuel gas consumption, net oxygen consumption, and the overall operational expenditures (OPEX) of the methanol plant.

The present invention proposes a configuration for methanol production utilizing a hydrogen-rich feedstock gas containing helium (like synthetic loop purge gas) and a carbon dioxide-rich feedstock gas, eliminating the need for oxygen (in autothermal reformers) and elevated pressure reforming for an elevated pressure syngas generation. Initially, mixture of a hydrogen-rich feedstock gas containing helium, a carbon dioxide-rich feedstock gas, and an inert methane-rich stream are de-pressurized in turboexpanders. Then, carbon dioxide conversion to carbon monoxide occurs at a low pressure in the presence of hydrogen and methane within a reverse water gas shift (RWGS) reactor. Notably, the presence of methane suppresses the methanation reactions, while a low pressure feed in the reverse water gas shift (RWGS) reactor helps reduce the cost of RWGS reactor mechanical design in a high-temperature. Subsequently, the produced carbon monoxide and residual hydrogen are re-pressurized in compressors and directed to series-connected methanol reactors for methanol synthesis.

The present invention proposes using solvent absorber columns (TEGDE absorber columns) between all of the methanol reactors. These columns uses a high-boiling inert solvent like tetraethylene glycol dimethyl ether (tetraglyme) (TEGDE) that selectively absorbs methanol and water. This configuration maximizes hydrogen conversion to methanol. In the process of separating methanol from methanol reactors outlet gases, solvent absorber columns achieve a higher methanol removal efficiency compared to the method of cooling and subsequent separation of the liquid methanol. This enhanced removal through absorption leads to increase a methanol conversion in the next methanol reactors. Additionally, absorption by TEGDE is more effective than refrigeration techniques for separating methanol when there is less pressure.

In the methanol production process, limitations inherent to the methanol production reaction at low hydrogen concentrations prevent the complete conversion of all hydrogen in the synthetic loop purge gas mixture. Therefore, the present invention proposes converting the remaining synthetic loop purge gas hydrogen to methane using an arrangement that simultaneously uses excess carbon dioxide gas as a feedstock and a heat removal medium for methanation reactions. This arrangement uses hydrogen to produce methane instead of the remaining hydrogen oxidation and producing steam, which reduces the side effect of deleting a synthetic loop purge gas as a main reformer fuel and reduces the consumption of oxygen as an oxidizing feedstock. The present invention also increases the carbon dioxide consumption of the present invention process, which helps to reduce the carbon dioxide footprint.

For helium purification, the outlet gas of the methanation section is routed to a carbon dioxide recovery membrane unit that separates carbon dioxide gas from other gases. In the next step, the remaining gases pass through a cryogenic nitrogen rejection unit, where methanol is injected in the gases to prevent hydrate formation and then the mixture is cooled in a first coldbox. After that, the gases are washed by cryogenic temperature liquid methanol streams in a first carbon dioxide absorber tower for water and carbon dioxide removal. After that, hydrogen, nitrogen, carbon monoxide and helium are cooled and separated from liquid methane by a second coldbox and a drum. Nitrogen and carbon monoxide are separated from hydrogen and helium by a recycled high-purity liquid methane stream in a first methane absorber tower. Then, remain methane gas and hydrogen gas are converted to water, carbon monoxide and carbon dioxide in a hydrogen catalytic combustion reactor.

Produced water and carbon dioxide in the hydrogen catalytic combustion reactor is absorbed by another cryogenic temperature liquid methanol stream in a second carbon dioxide absorber tower. Then, Nitrogen and carbon monoxide are separated from helium by another recycled high-purity liquid methane stream in a second methane absorber tower. Finally, methane is separated from helium by a recycled high-purity liquid propane stream in a propane absorber tower. Finally, remaining accompanying gases are separated from helium in a cryogenic adsorption unit. Cryogenic adsorption on activated carbon beds separates methane, nitrogen and other unwanted gases from helium.

The present invention proposes an arrangement to removes most methane and nitrogen gases from unreacted carbon dioxide in the carbon dioxide recovery membrane unit and the first carbon dioxide absorber tower in the cryogenic nitrogen rejection unit. Then, the unreacted carbon dioxide plus a fresh carbon dioxide feedstock are compressed and recirculated to the methanation section and the reverse water gas shift (RWGS) section.

### BRIEF DESCRIPTION OF DRAWINGS

The present invention will hereinafter be described in conjunction with the appended figures wherein like numbers denote same features throughout and wherein:

FIG. 1 is a block diagram illustrating of a system for processing a hydrogen-rich feedstock gas containing helium stream, a carbon dioxide-rich feedstock gas stream, and an inert methane-rich stream to obtain a high-purity helium gas product, while simultaneously producing a methanol-water liquid mixture and a methane-rich fuel product.

FIG. 2, FIG. 3, FIG. 4, FIG. 5 and FIG. 6 provide a process of an embodiment of the invention.

### DETAILED DESCRIPTION OF THE INVENTION

The following detailed description of the invention exemplifies preferred embodiments of the invention. It is not intended to restrict the invention's scope, applicability, or configuration. Rather, this detailed description of the invention provides a comprehensive disclosure, enabling those skilled in the art to implement the preferred embodiments. Deviations in the functionality and arrangement of elements may be made without infringing the spirit and scope of the invention as defined by the appended claims.

The term “comprising” as used in the claims does not exclude other elements or steps. The term “a” or “an” as used in the claims does not exclude a plurality. A “unit” or other means may fulfill the functions of several units or means recited in the claims. The terms “gas” or “gases” may be used interchangeably throughout the claims, and do not imply the presence of 100.0 mole percent concentration pure gas. The terms “liquid” or “liquids” may be used interchangeably throughout the claims, and do not imply the presence of 100.0 mole percent concentration pure liquid.

Whilst endeavoring in the foregoing specification to draw attention to those features of the invention believed to be of particular importance it should be understood that the Applicant claims protection in respect of any patentable feature or combination of features hereinbefore referred to and/or shown in the drawings whether or not particular emphasis has been placed thereon.

The term “rich” (used in conjunction with “lean” for the opposite) refers to a stream that has a higher mole fraction (or mole percent) of the indicated component compared to the original stream from which it was formed, or has a mole percent concentration greater than 50.0%. In a reaction, the term “excess” refers to a component present in a stream in a greater amount than what is stoichiometrically required. The term “high-purity” refers to a stream that has a mole percent concentration of the indicated component greater than 99.1%.

Within a process flow, the terms “downstream” and “upstream” designate the relative positions of a unit or equipment based on the intended direction of process fluid movement. The subsequent device is considered downstream if the process fluid is designed to flow from the initial device to the second one. In the presence of recycle streams, downstream and upstream designations reference the initial, non-recycled path of the process fluid.

This invention uses the term “column” or “tower” to refer to either a single one or a group of them. These columns or towers can be arranged in a line (series) or side-by-side (parallel). Each column or tower can be made up of one or more sections that hold trays and/or packing materials.

The term “catalytic oxidation” refers to a purification method for helium streams that eliminates hydrogen and/or methane. This process involves a chemical reaction with oxygen using a catalyst bed.

The term “low pressure” refers to a stream that has a pressure less than or equal to 20.0 barg. The term “high pressure” refers to a stream that has a pressure greater than 20.0 barg and a pressure less than 40.0 barg. The term “high-high pressure” refers to a stream with a pressure exceeding or equal to 40.0 barg.

The term “cryogenic temperature” or “cryogenic” refers to a stream or equipment or a unit that has a temperature less than or equal to −30.0° C.

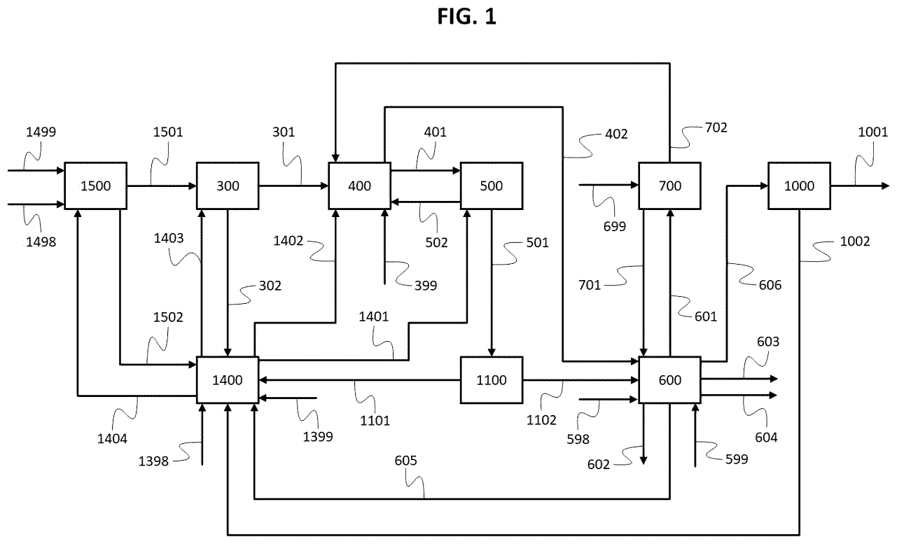

A detailed description of the claimed process and its associated apparatus is provided in conjunction with FIG. 1.

The term “demineralized water” refers to a stream that has a mole percent concentration of water component greater than 99.9% and has conductivity values less than 1.0 μS/cm conductivity.

The term “TEGDE” refers to tetraethylene glycol dimethyl ether (tetraglyme)

Trace quantities of helium are typically found within the natural gas deposits. Petrochemical plants utilizing the natural gas as a feedstock often employ high-temperature processes, such as steam reforming and autothermal reforming, to convert methane, the primary component of the natural gas, into carbon monoxide and hydrogen. This combined gas mixture, consisting of carbon monoxide and hydrogen, is called syngas.

Within a synthesis gas loop, the syngas repeatedly interacts with catalysts, enabling the production of diverse chemical species based on a specific catalyst employed. Examples include methanol, dimethyl ether (DME), and heavier hydrocarbons through the Fischer-Tropsch process. However, helium of the natural gas and other inert gases like nitrogen tend to accumulate within the loop. To maintain process integrity, a portion of the circulating syngas, known as synthetic loop purge gas, is periodically discharged from the loop to remove these inert components.

Compared to a helium content in a plant's natural gas feedstock, a concentration of helium within the synthetic loop purge gas is significantly higher. Due to its richness in hydrogen, the synthetic loop purge gas often serves as a fuel source for heating main reformers, contributing upwards of 50.0% of the required fuel gas energy in, for instance, methanol plants. Notably, converting the synthetic loop purge gas to alternative fuels does not translate to energy savings. Modern methanol plants, instead, employ hydrogen recovery units (HRU) that extract and reintegrate the extracted hydrogen with a carbon dioxide feedstock back into the process. While this configuration enhances methanol production, it concomitantly leads to increased net fuel gas consumption, oxygen consumption, and operational expenditures (OPEX) for the methanol plant.

According to the schematic in FIG. 1, the present invention provides systems and methods for obtaining a high-purity helium gas product (1001) and simultaneously producing a methanol-water liquid mixture (604) and a methane-rich fuel product (603) from a hydrogen-rich feedstock gas containing helium stream (1499) (like synthetic loop purge gas), a carbon dioxide-rich feedstock gas stream (1398), and an inert methane-rich gas stream (1498) in petrochemical plants that use the natural gas as a feedstock, such as methanol, dimethyl ether (DME), or heavy hydrocarbons (Fischer-Tropsch process) plants. This configuration allows many existing petrochemical plants to produce helium as a by-product, which is an expensive, low-supply, and limited-source substance.

This configuration optimizes the generation of high-value products (helium and methanol) and minimizing the utilization of hydrogen-rich feedstock gas containing helium as an alternative to fuel gas. This economic incentive encourages the elimination of function of hydrogen-rich feedstock gas containing helium as a fuel gas. Furthermore, the present invention introduces systems and techniques for employing carbon dioxide as a feedstock.

According to the schematic in FIG. 1, the hydrogen-rich feedstock gas containing helium stream (1499), comprising hydrogen, helium, and other gaseous components is injected into a reverse water gas shift unit (1500). Also the inert methane-rich gas stream (1498) along with a high-high pressure recycled carbon dioxide-rich gas stream (1404) from a solvent recovery unit (1400) are injected into the unit 1500. The high-high pressure recycled carbon dioxide-rich gas stream (1404) is a first portion of a compressed overhead gases from a first water absorber column.

A molar ratio of hydrogen to carbon dioxide ranges from 1.0 to 8.0 in the mixture of the hydrogen-rich feedstock gas containing helium stream (1499), the inert methane-rich gas stream (1498), and the high-high pressure recycled carbon dioxide-rich gas stream (1404), depends on the type of reverse water gas shift catalysts, a reverse water gas shift reactor operating temperature, and a reverse water gas shift reactor operating pressure. Also a molar ratio of carbon dioxide to methane ranges from 1.0 to 4.5 in the mixture of the hydrogen-rich feedstock gas containing helium stream (1499), the inert methane-rich gas stream (1498), and the high-high pressure recycled carbon dioxide-rich gas stream (1404), depends on the methane selectivity of the reverse water gas shift reaction. A mole percent of carbon dioxide in the mixture of stream 1499, stream 1498 and stream 1404 exceeds 10.0%. A mole percent of methane in the mixture of stream 1499, stream 1498 and stream 1404 exceeds 10.0%.

To prevent condensate formation, the streams 1499, 1498, and 1404 are preheated in the unit 1500 before pressure reduction in a downstream turboexpander. The hot gases enter a turboexpander for pressure reduction. Subsequently, they pass through a series of heat exchangers and a fired heater to achieve a desired reaction temperature before injection into a catalyst-filled reactor where the reverse water gas shift (RWGS) reaction occurs.

CO2+H2→CO+H2O

The RWGS reactor outlet gases are cooled. Then, the cooled gases enter a gas-liquid separator. a liquid stream (1502) from the separator, containing water liquid and some dissolved gases, is directed to the solvent recovery unit (1400) to exploit its water liquid content for methanol absorption. The separated gases from the gas-liquid separator are compressed in compressors. Notably, These compressors could be coupled with the aforementioned turboexpander to utilize its generated power. Compressed gases through stream 1501 exit the unit 1500 and are sent to a methanol production unit (300).

In the unit 1500, the streams 1498, 1499 and 1404 enter a reactor containing a catalyst bed, where the reverse water gas shift (RWGS) reaction takes place.

CO2+H2→CO+H2O

During the conversion of carbon dioxide and hydrogen using the RWGS process, the methanation reactions act as unwanted competitors.

CO2+4H2→CH4+2H2O

CO+3H2→CH4+H2O

This competition presents a significant hurdle when developing catalysts specifically for RWGS. The choice between forming carbon monoxide or methane during the conversion of carbon dioxide and hydrogen is highly influenced by pressure. Higher pressure favors methane production as it involves fewer gas molecules, following Le Chatelier's principle. To reduce methane production in the RWGS reactor, the present invention proposes mixing the inert methane-rich gas stream (1498) with the streams 1499 and 1404 and also simultaneously lowering inlet stream pressure of the reverse water gas shift reactor to a low pressure by the turboexpander.

The hydrogen-rich feedstock gas containing helium stream (1499) is produced in a methanol, dimethyl ether (DME), heavy hydrocarbons (Fischer-Tropsch process) or similar plant and does not typically require desulfurization. However, if a hydrogen-rich feedstock gas containing helium stream from other facilities contains trace amounts of sulfur-containing compounds, it is recommended to incorporate two different types of reactors at the beginning of unit 1500, following the heating of streams 1498, 1499 and 1404. The first reactor would be designed to specifically convert these sulfur compounds into hydrogen sulfide (H2S). Then, the second reactor, equipped with an adsorbent bed, would effectively remove the generated H2S.

If the hydrogen-rich feedstock gas containing helium stream (1499) contains methanol, which could be cracked at a high temperature, a water wash column may be used at the beginning of the unit 1500 to prevent coke forming and the RWGS catalyst deactivation. If the hydrogen-rich feedstock gas containing helium stream (1499) contains other heavy hydrocarbons that could be cracked at a high temperature, parallel RWGS reactors can be employed for continuous operation, with one reactor on-stream for processing while the other undergoes regeneration (steam decoking). Also steam reforming on nickel-based catalysts with hydrogen (adiabatic pre-reformers) presents a potentially cost-effective method for the removal of heavy hydrocarbons.

According to the schematic in FIG. 1, the compressed gases through stream 1501 are sent from the unit 1500 to the unit 300. First, in order to prepare them for the methanol production reaction, the temperature of the gases in stream 1501 is adjusted to an appropriate value. Then, they enter a first methanol reactor. The following reactions occur on a catalytic bed:

CO+2H2→CH3OH

CO2+3H2→CH3OH+H2O

To reduce methanol and water vapor in gases entering downstream methanol reactors and prevent them from hindering methanol production, the present invention proposes using solvent absorber columns (TEGDE absorber columns) between all the methanol reactors. These columns use a high-boiling inert solvent like tetraethylene glycol dimethyl ether (tetraglyme) (TEGDE) that selectively absorbs methanol and water. When a methanol reactor's outlet gas comes in contact with the TEGDE, methanol and water molecules are absorbed.

In the process of separating methanol from a methanol reactor outlet gases, TEGDE absorber columns achieve a higher methanol removal efficiency compared to a method of cooling and subsequent separation of the liquid methanol. This enhanced removal through absorption leads to increase a methanol conversion in the next methanol reactors. Additionally, absorption by TEGDE is more effective than refrigeration techniques for separating methanol when there is less pressure.

The first methanol reactor's outlet gas comes in contact with the TEGDE from a methanol-lean solvent stream (1403) in a TEGDE absorber column. The exiting gas temperature from the TEGDE absorber column is adjusted to an appropriate value for the methanol production reaction, and subsequently they enter a second methanol reactor. The following reactions occur on a catalytic bed:

CO+2H2→CH3OH

CO2+3H2→CH3OH+H2O

To design methanol reactors with maximum conversion, the present invention proposes increasing the methanol reactors length and using series-connected methanol reactors.

During methanol synthesis from carbon monoxide and hydrogen, a competing reaction, methanation reaction, poses a challenge. While the hydrogen-rich feedstock gas containing helium stream (1499) often contains sufficient methane to suppress this undesired pathway, the present invention proposes an additional strategy to further minimize methane production within the methanol reactors. The process involves the controlled introduction of the inert methane-rich gas stream (1498) into the streams 1499 and 1404 in such a way that a mole percent of methane in the first methanol reactor feed gas exceeds 10.0%. To design a methanol reactor with maximum conversion, a mole percent of carbon dioxide in the first methanol reactor feed gas exceeds 10.0%.

The methanol-rich solvent from the bottom of TEGDE absorber columns is sent to the solvent recovery unit (1400) through stream 302. In the unit 1400, in a reboilers of a methanol stripper tower, the methanol-rich solvent undergoes heating to remove methanol and water as a gaseous stream (stripped gases), resulting in a solvent with a low methanol and water content (methanol-lean solvent). Preventing its loss, Vapor TEDGE condenses into liquid TEDGE in a partial condenser at the top of methanol stripper tower and condensates return to the methanol stripper tower. Then, the methanol-lean solvent is cooled in a heat exchanger and pumped to the top of TEGDE absorber columns in the unit 300 through stream 1403.

According to the schematic in FIG. 1, in the unit 1400, after passing through the partial condenser, the stripped gases from the top of methanol stripper tower enter the bottom of first water absorber column. Also a degassing drum vent gas from a cryogenic nitrogen rejection unit (600) through stream 605, a tail gas from a cryogenic adsorption unit (1000) through stream 1002, the carbon dioxide-rich feedstock gas stream (1398), and separated carbon dioxide in a carbon dioxide recovery membrane unit (1100) through stream 1101 enter the bottom of first water absorber column. In the first water absorber column, methanol in the stripped gases is absorbed through a second portion of a liquid demineralized water (stream 1399). Stream 1399 is injected from the top of first water absorber column onto the packing inside the tower. Water liquid from the reverse water gas shift unit (1500) through stream 1502 is sent to the top of first water absorber column to facilitate methanol absorption. Stream 1402, a liquid mixture of water and methanol, exits the bottom of first water absorber column and is sent to a second water absorber column in a methanol absorption unit (400) for mixing with other methanol products.

According to the schematic in FIG. 1, overhead gases from the first water absorber column are compressed in a multi stage compressor with interstage coolers and separator drums. The first portion of the compressed overhead gases of the first water absorber column, the high-high pressure recycled carbon dioxide-rich gas stream, is sent to the unit 1500 through stream 1404 and a second portion of the compressed overhead gases of the first water absorber column, a high pressure recycled carbon dioxide-rich gas stream, is sent to a methane production unit (500) through stream 1401. A molar fraction of methanol in streams 1401 and 1404 is less than 1.0E-6.

The selection of the optimal product from a methanol reactor is contingent upon project-specific economic considerations. By adjusting the synthesis catalyst, the choice may fall between directly producing either methanol or dimethyl ether (DME) at the methanol reactors outlet. Additionally, for projects with limited feedstock capacity, the liquid-phase methanol process might offer a more favorable option compared to the gas-phase process. Furthermore, alternative processes such as heavy hydrocarbon production (Fischer-Tropsch process), ammonia production, and acetic acid production could also be viable depending on the project's specific needs and economic feasibility.

One alternative approach to maximize the yield of high-value products involves injecting the mixture of the streams 1499, 1404, and 1498 into an olefin production reactor containing Fe2O3 encapsulated in K2CO3 catalyst, for carbon dioxide conversion to olefin by a tandem mechanism at temperature 350.0° C. This would be followed by cryogenic temperature separating olefin products and sending the remaining mixture to the unit 1500. The major economic challenge of this method is the cost of storage and distillation of olefin products. If an olefin unit or an oil refinery is located nearby, using the mentioned method is appropriate. An olefin unit usually has a fuel hydrogen stream, but this fuel stream does not contain helium. An olefin unit fuel hydrogen stream could be used as a feedstock of the unit 1500.

According to the schematic in FIG. 1, the gases exiting the second methanol reactor are sent from the unit 300 to the methanol absorption unit (400) through stream 301. First, the gas products from the second methanol reactor are cooled. Then, they enter the second water absorber column from bottom. The second water absorber column separates liquid and gas phases, and methanol in the gas phase is absorbed by a first portion of the liquid demineralized water (stream 399) within the second water absorber column. Stream 399 is injected from the top of second water absorber column onto the packing inside the tower.

According to the schematic in FIG. 1, water liquid from the methane production unit (500) through stream 502 and water liquid from a catalytic oxidation unit (700) through stream 702 are pumped to the unit 400. In the unit 400, the streams 502 and 702 are injected into the top of second water absorber column to facilitate methanol absorption. In order to be mixed in other methanol products, the liquid mixture of water and methanol, originating from the unit 1400 via stream 1402, enters the bottom of second water absorber column in the unit 400. Stream 402, a liquid raw methanol, is sent from the bottom of second water absorber column to the cryogenic nitrogen rejection unit (600). The exhaust gases from the second water absorber column are sent to the unit 500 through stream 401. Deep methanol removal is necessary for downstream methanation reactors because methanol cracking at a high temperature can form coke, which reduces catalyst activity. A molar fraction of methanol in stream 401 is less than 1.0E-6.

According to the schematic in FIG. 1, the exhaust gases from the unit 400 through stream 401 and the high pressure recycled carbon dioxide-rich gas stream from the unit 1400 through stream 1401 are sent to the unit 500. In the unit 500, the inlet gases are mixed. First, the gas mixture temperature is increased to an appropriate reaction temperature. Then, the gases enter series-connected methanation reactors. Within the methanation reactors of the unit 500, hydrogen, carbon monoxide and carbon dioxide gases are converted to methane and Water on a catalytic bed through the Sabatier reactions.

CO2+4H2→CH4+2H2O

CO+3H2→CH4+H2O

To prevent the methanation reactions heat from causing an excessive increase in the temperature of the methanation reactors outlet, the mole percent of carbon dioxide at a first methanation reactor inlet, reaching up to 40.0%, exceeds the stoichiometric amount required for the methanation reactions. Heat from the methanation reactions is transferred to water liquid in heat exchangers located after each methanation reactor for steam generation. If coking damages catalysts activity that are used in the methanation reactors, parallel methanation reactors can be employed for continuous operation in the unit 500, with one methanation reactor on-stream for processing while the other undergoes regeneration (steam decoking).

According to the schematic in FIG. 1, in the unit 500, the last methanation reactor outlet hot gases are cooled and then they are sent to a drum where they are separated into liquids and gases. The liquid from the drum through stream 502 is sent to the unit 400 to use its water liquid to facilitate methanol absorption. The obtained gases from the drum are sent to the carbon dioxide recovery membrane unit (1100) through stream 501.

According to the schematic in FIG. 1, the gases through stream 501 enter the carbon dioxide recovery membrane unit (1100), where they undergo a membrane separation process by DDR-type zeolite membranes. This process facilitates the separation of carbon dioxide from hydrogen, helium, nitrogen, methane, carbon monoxide and other gases. Separated carbon dioxide gas is sent to the unit 1400 through stream 1101 for pressurization. Hydrogen, helium, nitrogen, methane, carbon monoxide and other gases are sent to the cryogenic nitrogen rejection unit (600) through stream 1102.

According to the schematic in FIG. 1, hydrogen, helium, nitrogen, methane, carbon monoxide and other gases enter the cryogenic nitrogen rejection unit (600) from the unit 1100 through stream 1102. Stream 402, the liquid raw methanol, is sent from the bottom of second water absorber column to the unit (600). In the unit 600, a first portion of the liquid raw methanol is injected in the stream 1102 inlet gases to prevent hydrate formation in cryogenic temperatures of downstream equipment and then the mixture is cooled in a first coldbox. The obtained gas and liquid from the first coldbox are sent to a second reboiler of a propane distillation tower and then they are sent to a first reboiler of a nitrogen stripper tower and after that they are sent to a bottom of first carbon dioxide absorber tower, where the obtained gas is washed by a cryogenic temperature liquid methanol stream and a first portion of a recycled cryogenic temperature liquid methanol stream on the packed beds for water and carbon dioxide removal.

According to the schematic in FIG. 1, in the unit 600, the first carbon dioxide absorber tower outlet gas is sent to a second reboiler of the nitrogen stripper tower and then it is sent to a second coldbox to cool by a liquid nitrogen refrigerant (stream 599) and other cold streams. After that, it is sent to a drum to separate gaseous hydrogen, nitrogen, methane, carbon monoxide, and helium from liquid methane and other liquids. The liquid methane and other liquids are sent to a second condenser of the nitrogen stripper tower, the second coldbox, and the first coldbox to warm up and then they mix in a heated non-condensable off-gas from a reflux drum of the nitrogen stripper tower and a heated waste gas from a reflux drum of the propane distillation tower. This mixture, the methane-rich fuel product, is sent to petrochemical plants' fuel gas network through stream 603. The gaseous hydrogen, nitrogen, carbon monoxide, and helium are sent to a first methane absorber tower where nitrogen and carbon monoxide are separated from hydrogen and helium by a first portion of a recycled high-purity liquid methane stream.

According to the schematic in FIG. 1, in the unit 600, the first methane absorber tower outlet gases are sent to the second coldbox and the first coldbox to warm up and then they are sent to the catalytic oxidation unit (700) through stream 601. The liquid from the bottom of first methane absorber tower is sent to a first condenser of the nitrogen stripper tower and then it is sent to the top of nitrogen stripper tower where nitrogen, oxygen, and carbon monoxide are stripped from liquid methane on the packed beds or trays to produce a high-purity liquid methane from the bottom of nitrogen stripper tower. A first portion of the high-purity liquid methane from the bottom of nitrogen stripper tower is sent to the first reboiler of the nitrogen stripper tower to adjust nitrogen impurity and oxygen impurity in the high-purity liquid methane from the bottom of nitrogen stripper tower. Also a second portion of the high-purity liquid methane from the bottom of nitrogen stripper tower is sent to the second reboiler of the nitrogen stripper tower to adjust nitrogen impurity and oxygen impurity in the high-purity liquid methane from the bottom of nitrogen stripper tower.

According to the schematic in FIG. 1, in the unit 600, a third portion of the high-purity liquid methane from the bottom of nitrogen stripper tower is pumped to the second coldbox to cool down and then it is send to a second liquid-liquid heat exchanger. In next step, it is sent to the second coldbox from the second liquid-liquid heat exchanger to produce the recycled high-purity liquid methane stream. After that, the first portion of the recycled high-purity liquid methane stream is routed from the second coldbox to the top of first methane absorber tower. The nitrogen stripper tower overhead gases are sent to the first condenser of the nitrogen stripper tower and after that they are sent to the second condenser of the nitrogen stripper tower. The second condenser of the nitrogen stripper tower outlet gas and liquid are send to the reflux drum of the nitrogen stripper tower where they are separated into liquids and the non-condensable off-gas. The liquid from the reflux drum of the nitrogen stripper tower is sent back to the nitrogen stripper tower. The non-condensable off-gas is send to the first coldbox to warm up and then it mix in the other gaseous methane source. This mixture, the methane-rich fuel product, is sent to petrochemical plants' fuel gas network through stream 603.

According to the schematic in FIG. 1, a liquid nitrogen refrigerant, stream 599, vaporizes in the second coldbox and cools down hot streams and then vaporized nitrogen is sent to the first coldbox to warm up and vent to a safe location through stream 602. A second portion of the liquid raw methanol is cooled by a second portion of first carbon dioxide absorber tower outlet liquid methanol in a first liquid-liquid heat exchanger and then it is cooled in the second liquid-liquid heat exchanger to produce the cryogenic temperature liquid methanol stream. The cryogenic temperature liquid methanol stream enters from the top of first carbon dioxide absorber tower for water and carbon dioxide removal.

According to the schematic in FIG. 1, in the unit 600, after heating in the first liquid-liquid heat exchanger, the second portion of first carbon dioxide absorber tower outlet liquid methanol is sent to the degassing drum where it is separated into liquids and gases. The degassing drum vent gas is sent to the unit 1400 through stream 605. The degassing drum outlet liquid methanol, the methanol-water liquid mixture, is sent to a methanol plant is located in the outside of the battery limit for methanol purification, through stream 604. A first portion of first carbon dioxide absorber tower outlet liquid methanol is pumped to the first coldbox to warm up and then it is sent to a drum where it is separated into liquids and gases. The obtained gases from the drum are sent to the degassing drum. The liquid from the drum is sent back to the first coldbox to cool down and to produce the recycled cryogenic temperature liquid methanol stream. The first portion of the recycled cryogenic temperature liquid methanol stream is sent to the top of first carbon dioxide absorber tower for water and carbon dioxide removal. A second portion of the recycled cryogenic temperature liquid methanol stream is sent to a second carbon dioxide absorber tower.

According to the schematic in FIG. 1, in the unit 600, the gaseous hydrogen, helium, methane and a few other gases exit from the top of first methane absorber tower and then they are sent to the second coldbox and the first coldbox to warm up and after that they are sent to the catalytic oxidation unit (700) through stream 601. First, gases of stream 601 are compressed in a compressor. The compressed gaseous are heated in a heat exchanger and then they are fed into a hydrogen catalytic combustion reactor, where a high-purity gaseous oxygen from stream 699 oxidize hydrogen and methane on a catalytic bed, according to the following reaction:

2H2+O2→2H2O

CH4+2O2→CO2+2H2O

CH4+O2→CO+2H2O

An excess oxygen gas through stream 699 and a catalyst type for the hydrogen catalytic combustion bed are selected in such way that a molar fraction of hydrogen in stream 701 is less than 5.0E-7. There is nitrogen in the outlet gases of hydrogen catalytic combustion reactor because the high-purity gaseous oxygen from stream 699 has nitrogen impurity.

According to the schematic in FIG. 1, in the unit 700, the hydrogen catalytic combustion reactor outlet hot gases are cooled and then they enter a drum where they are separated into liquids and gases. The liquid from the drum, which contains water liquid and some dissolved gases, is pumped to the unit 400 through stream 702 to use its water liquid for methanol absorption. The exit gas from the drum, which contains helium, methane, water, carbon dioxide, carbon monoxide, nitrogen, oxygen, and some other impurities is sent back from the unit 700 to the unit 600 through stream 701.

According to the schematic in FIG. 1, in the unit 600, a third portion of the liquid raw methanol is injected in the stream 701 inlet gases to prevent hydrate formation in cryogenic temperatures of downstream equipment and then the mixture is sent to a first reboiler of the propane distillation tower and after that it enters the bottom of second carbon dioxide absorber tower. The second portion of the recycled cryogenic temperature liquid methanol stream is sent to the top of second carbon dioxide absorber tower for water and carbon dioxide removal. A cryogenic temperature liquid methanol-rich stream is sent from a bottom of second carbon dioxide absorber tower to the bottom of first carbon dioxide absorber tower. The outlet gas from the top of second carbon dioxide absorber tower is send to a liquid propane heat exchanger and after that it is sent to a second methane absorber tower for nitrogen, oxygen, and carbon monoxide removal. A second portion of the recycled high-purity liquid methane stream is routed from the second coldbox to the top of second methane absorber tower where nitrogen, oxygen, and carbon monoxide are separated from helium.

According to the schematic in FIG. 1, in the unit 600, the liquid from the bottom of second methane absorber tower is sent to the top of nitrogen stripper tower. The second methane absorber tower outlet gases are sent to a propane absorber tower. A recycled high-purity liquid propane stream is routed from a second condenser of the propane distillation tower to the top of propane absorber tower where methane is separated from helium. The outlet gas from the top of propane absorber tower is send to the cryogenic adsorption unit (1000) through stream 606. A cryogenic temperature liquid propane-rich stream is sent from a bottom of propane absorber tower to the liquid propane heat exchanger and after that it is sent to a first condenser of the propane distillation tower and then it is sent to the propane distillation tower where methane is stripped from liquid propane on the packed beds or trays to produce a high-purity liquid propane from the bottom of propane distillation tower.

According to the schematic in FIG. 1, in the unit 600, the propane distillation tower overhead gases are sent to the first condenser of the propane distillation tower and the second condenser of the propane distillation tower. The second condenser of the propane distillation tower outlet gas and liquid are send to a reflux drum of the propane distillation tower where they are separated into liquids and the waste gas. The liquid from the reflux drum of the propane distillation tower is sent back to the top of the propane distillation tower. The waste gas is send to the first coldbox to warm up and then it mix in the other gaseous methane source. This mixture, the methane-rich fuel product, is sent to petrochemical plants' fuel gas network through stream 603.

According to the schematic in FIG. 1, in the unit 600, a first portion of the high-purity liquid propane from the bottom of propane distillation tower is sent to the first reboiler of the propane distillation tower to adjust methane impurity in the high-purity liquid propane from the bottom of propane distillation tower. Also a second portion of the high-purity liquid propane from the bottom of propane distillation tower is sent to the second reboiler of the propane distillation tower to adjust methane impurity in the high-purity liquid propane from the bottom of propane distillation tower. A third portion of the high-purity liquid propane from the bottom of propane distillation tower is pumped to the second coldbox to cool down and then it is send to the second condenser of the propane distillation tower to produce the recycled high-purity liquid propane. In next step, the recycled high-purity liquid propane is routed from the second condenser of the propane distillation tower to the top of propane absorber tower. A make-up high-purity liquid propane, stream 598, is injected in the top of propane distillation tower to compensate for gaseous propane in stream 603 and other losses.

According to the schematic in FIG. 1, the gases exiting the top of propane absorber tower enter the unit 1000 from the unit 600 through stream 606. Cryogenic adsorption on activated carbon beds separates methane, nitrogen and other unwanted gases from helium. Stream 1001 is the high-purity helium gas product. A mole percent of helium in stream 1001 is greater than 99.999%. A ratio of a helium content by mole in stream 1001 to a helium content by mole in stream 1499 is greater than 0.71. The activated carbon adsorption beds are designed with at least two parallel trains, each with a 100.0% capacity, so that one train is on stream for processing while the other undergoes regeneration. A portion of stream 606 could be heated and used as a regeneration gas. According to the schematic in FIG. 1, during the regeneration process, the adsorbed waste materials by the activated carbon beds are released as the tail gas and send to the bottom of first water absorber column in the unit 1400 through stream 1002 for helium loss prevention.

All of the following configuration could be selected based on technical or commercial issues. It is possible that the first coldbox merge with the second coldbox in the unit 600. It is possible to use one methanation reactor instead of series-connected methanation reactors in the unit 500. It is possible to remove compressor in the unit 700, If the unit 700 inlet pressure is enough for combustion pressure of hydrogen and downstream units requirements. It is possible that the activated carbon adsorption beds to be designed with three parallel trains in the unit 1000, each with a 100.0% capacity, so that one train is on stream for processing while the other undergoes regeneration and the third undergoes cool down. It is possible that a liquid-liquid separator to be installed between the first liquid-liquid heat exchanger and the second liquid-liquid heat exchanger to remove remaining TEGDE from methanol at low temperature.

### DETAILED DESCRIPTION OF THE DRAWINGS

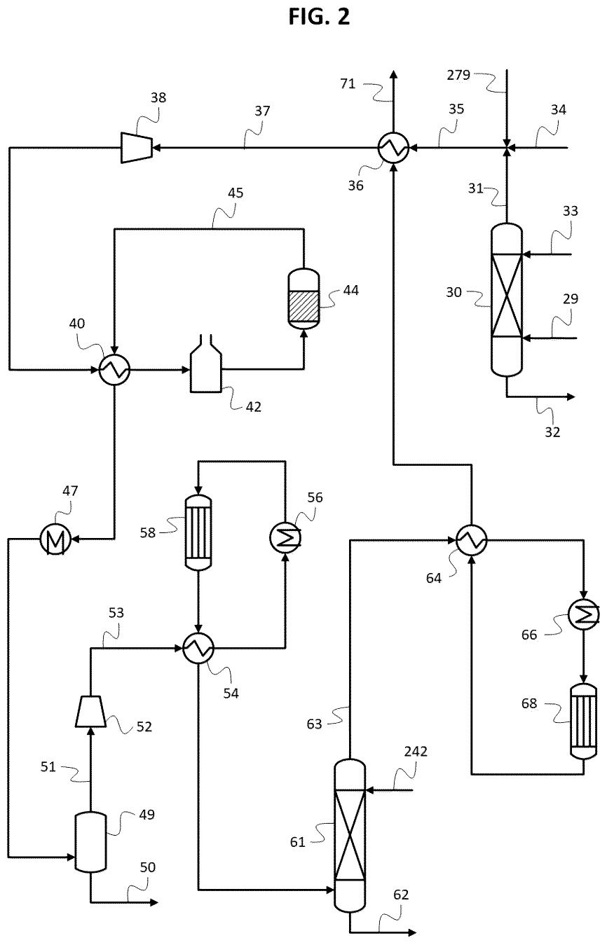

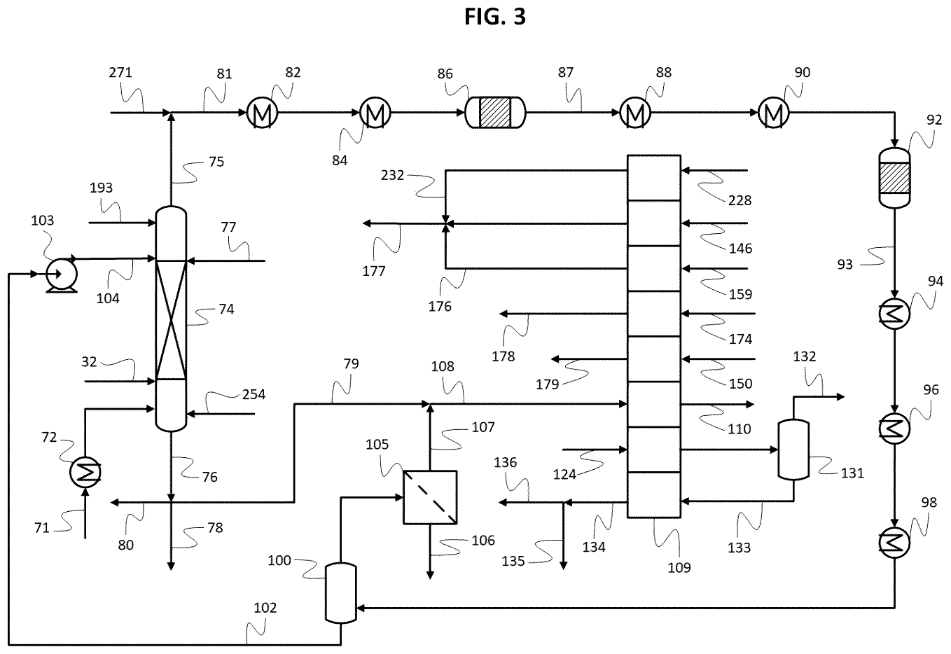

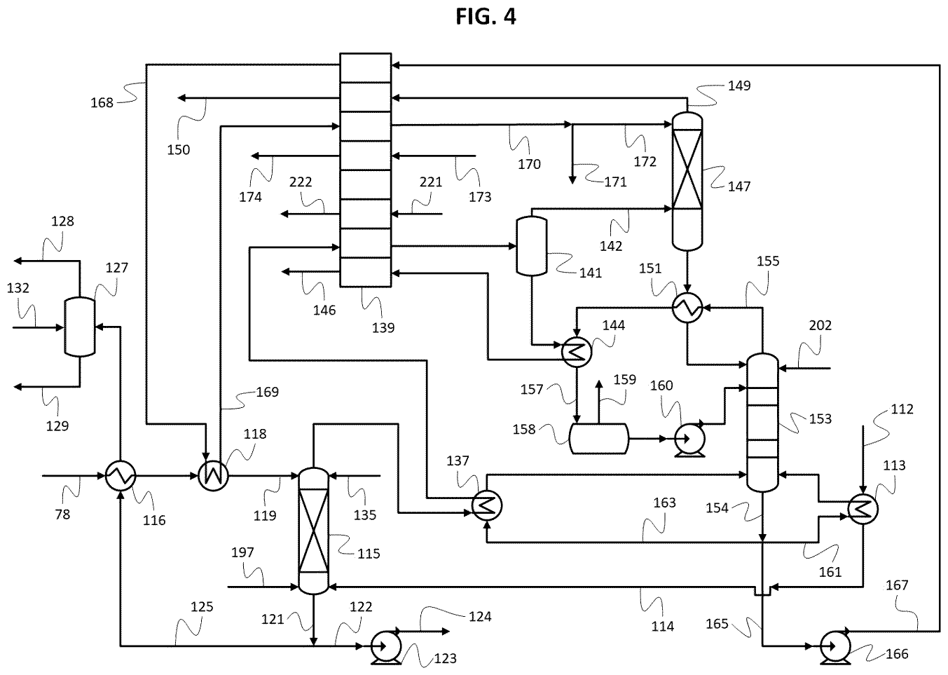

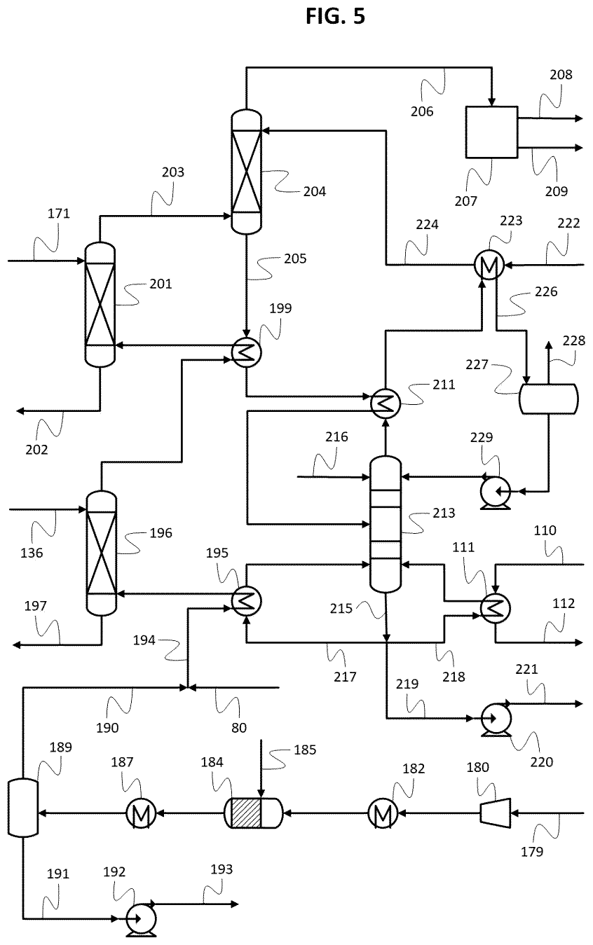

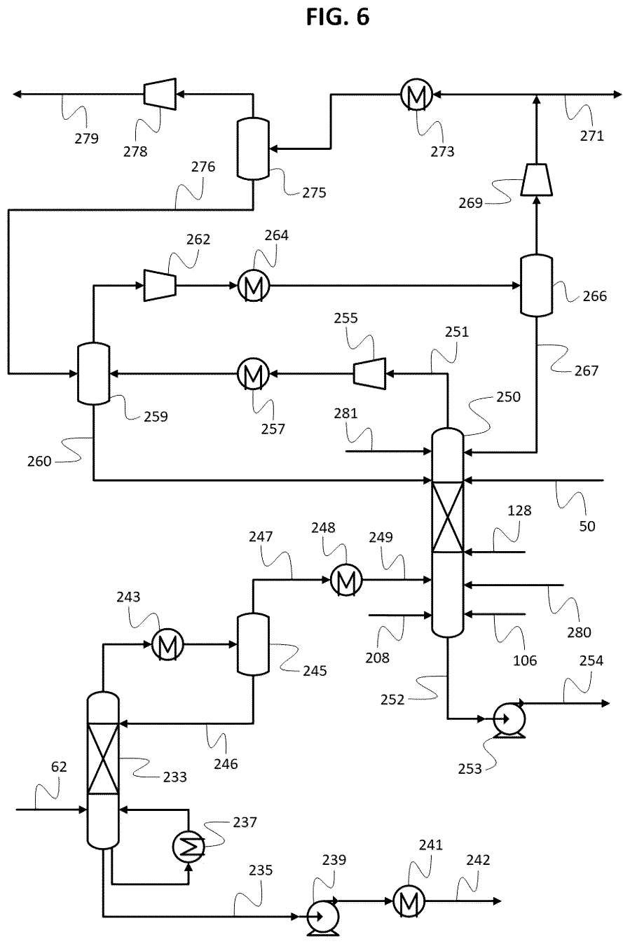

The systems and methods of the present invention may be further understood with reference to FIG. 2, FIG. 3, FIG. 4, FIG. 5, and FIG. 6, and the descriptions thereof that follow. It should be noted that the figures are simplified flow diagrams and, in some instances, do not show various pieces of auxiliary equipment, such as valves and control systems. Because one having ordinary skill in the art would recognize easily the need for and location of Such auxiliary equipment, its omission is appropriate and facilitates the simplification of the figures.

FIG. 2, FIG. 3, FIG. 4, FIG. 5, and FIG. 6 make an design method for the present invention and are parts of the present invention but they do not limit design methods scope for the present invention. For example, economically motivated changes to the number of equipment in the units, heat exchange between cold and hot streams (Pinch Technology), or deletion of equipment or units may be considered without limiting the scope of the present invention's design methods.

Fluid streams and equipment common to more than one figure or embodiment are identified by the same reference numerals in each figure.

According to FIG. 2, FIG. 3, FIG. 4, FIG. 5, and FIG. 6 stream 29, which is a hydrogen-rich feedstock gas containing helium (like synthetic loop purge gas) enters the system as the main feedstock. Stream 29 typically has a pressure of 80.0 barg, a temperature of 45.0° C., and mole percent of compositions: 70.0% hydrogen, 3.0% carbon monoxide, 7.0% carbon dioxide, 9.0% methane, 10.0% nitrogen, 0.5% methanol, 0.3% argon, and about 0.2% helium. Stream 29 contains methanol that could be cracked at a high temperature so a water wash column should be used to prevent coke forming and a downstream catalyst deactivation. Stream 29 is fed to a third water absorber column 30. Within the third water absorber column 30, methanol is absorbed by liquid demineralized water entering from the top of third water absorber column 30 through stream 33 on the packed bed. Subsequently, liquid mixture of water and methanol exits the bottom of third water absorber column 30 and is sent to a second water absorber column 74 via stream 32.

Stream 31, the third water absorber column 30 outlet gases, is combined with a high-high pressure recycled carbon dioxide-rich gas stream 279 so that the molar ratio of hydrogen to carbon dioxide ranges 1.0 to 8.0 in stream 35, depends on a catalyst type, an operating temperature, and an operating pressure in a reverse water gas shift reactor 44. Also stream 31 is mixed in a inert methane-rich gas stream 34 to achieve a molar ratio of carbon dioxide to methane ranges from 1.0 to 4.5 in stream 35, depends on the methane selectivity of the reverse water gas shift reaction. A mole percent of carbon dioxide in stream 35 exceeds 10.0%, and a mole percent of methane in stream 35 exceeds 10.0%.

The gas of stream 35 is heated to 90.0° C. in a steam heat exchanger 36. Then, the hot gases enter a turboexpander 38 through stream 37 to reduce their pressure to 10.0 barg. The outlet gases of turboexpander 38 are heated to 650.0° C. in a heat exchanger 40 and a furnace 42. To reduce investment costs, the furnace 42 can be constructed as a part of a main reformer of petrochemical plants or can be combined with heat exchangers of methanation section (88 and 94). Then, the outlet hot gases of furnace 42 enter the reverse water gas shift reactor 44 where the reverse water gas shift (RWGS) reaction occurs on the 1.0% Cu/β-Mo2C catalyst. The reaction on the catalyst bed is as follows:

CO2+H2→CO+H2O

During the conversion of carbon dioxide and hydrogen using the RWGS process, the methanation reactions act as unwanted competitors.

CO2+4H2→CH4+2H2O

CO+3H2→CH4+H2O

This competition presents a significant hurdle when developing catalysts specifically for RWGS. The choice between forming carbon monoxide or methane during the conversion of carbon dioxide and hydrogen is highly influenced by pressure. Higher pressure favors methane production as it involves fewer gas molecules, following Le Chatelier's principle. To reduce methane production in the RWGS reactors, the present invention proposes mixing the inert methane-rich gas stream 34 with the main feeds Streams and also simultaneously reduction the RWGS reaction pressure.

Following their exit from the RWGS reactor 44, the hot gases undergo cooling through the heat exchanger 40 and a water-cooled heat exchanger 47, achieving a final temperature of 45.0° C. It is possible to implement an air cooler before the water-cooled heat exchanger 47 (not shown for simplicity of the figure). Then, the generated water liquid and gas are separated in a drum 49. The separated water liquid in the drum 49 is sent to a first water absorber column 250 through stream 50, and the separated gases enter a compressor 52 through stream 51. The turboexpander 38 could be coupled to the compressor 52. Based on compressor type selection and compressor speed selection, 70.0%-90.0% of stream 37 pressure could be recovered. Water production and then its removal help pressure recovery. Also it is possible to implement a heat exchanger and a drum after compressor 52 (not shown for simplicity of the figure) to remove more water liquid at a higher pressure.

The gases from discharge of compressor 52 enter a heat exchanger 54 through stream 53. The gases temperature in the heat exchanger 54 and a steam heat exchanger 56 is raised to 230.0° C. and then the gases enter a first methanol reactor 58, where the methanol synthesis reaction takes place on the copper-zinc-alumina (Cu/ZnO/Al2O3) catalyst. The reactions on the catalyst bed are as follows:

CO+2H2→CH3OH

CO2+3H2→CH3OH+H2O

The heat of reaction in the first methanol reactor 58 is absorbed by converting water liquid to steam in the shell side of first methanol reactor 58. To design methanol reactors with maximum conversion, the present invention proposes increasing methanol reactors length and using series-connected methanol reactors.

During methanol synthesis from carbon monoxide and hydrogen, a competing reaction, methanation reaction, poses a challenge. While feedstock often contains sufficient methane to suppress this undesired pathway, the present invention proposes an additional strategy to further minimize methane production within the methanol production reactor. The process involves the controlled introduction of the inert methane-rich gas stream (30) into the primary feedstock streams.

To reduce methanol and water vapor in the gases entering downstream methanol reactors and prevent them from hindering methanol production, the present invention proposes using solvent absorber columns (TEGDE absorber columns) between all of the methanol reactors. These columns uses a high-boiling inert solvent like tetraethylene glycol dimethyl ether (tetraglyme) (TEGDE) that selectively absorbs methanol and water. When a methanol reactor's outlet gas comes in contact with the TEGDE, methanol and water molecules are absorbed. In the process of separating methanol from a methanol reactor outlet gases, TEGDE absorber columns achieve a higher methanol removal efficiency compared to a method of cooling and subsequent separation of the liquid methanol. This enhanced removal through absorption leads to increase a methanol conversion in the next methanol reactors. Additionally, absorption by TEGDE is more effective than refrigeration techniques for separating methanol when there is less pressure.

The hot gases from the first methanol reactor 58 undergo cooling through the heat exchanger 54. Then, they enter a TEGDE absorber column 61 to contact with a methanol-lean solvent through stream 242 that selectively absorbs methanol and water. After that, a methanol-rich solvent is sent to a methanol stripper tower 233 through stream 62.

The gases from the top of TEGDE absorber column 61 enter a heat exchanger 64 through stream 63. Flowrate of stream 242 control temperature of stream 63 less than 50.0° C. to prevent loss of TEGDE. After exiting the top of TEGDE absorber column 61, the gases temperature in the heat exchanger 64 and a steam 66 is raised to 230.0° C. and then the gases enter a second methanol reactor 68, where the methanol synthesis reaction takes place on the copper-zinc-alumina (Cu/ZnO/Al2O3) catalyst. The reactions on the catalyst bed are as follows:

CO+2H2→CH3OH

CO2+3H2→CH3OH+H2O

The heat of reaction in the second methanol reactor 68 is absorbed by converting water liquid to steam in the shell side of second methanol reactor 68.

The hot gases exiting the second methanol reactor 68 are cooled by the heat exchangers 64 and 36. Following their exit from the heat exchanger 36 through stream 71, the cooled gases undergo further cooling through a water-cooled heat exchanger 72, achieving a final temperature of 45.0° C. Then they enter the bottom of second water absorber column 74. Within the second water absorber column 74, the produced methanol is absorbed on the packed bed by the water liquid entering from the top of tower through streams 77, 193, and 104. Stream 77 contains liquid demineralized water. Stream 76, a liquid raw methanol, exit from the bottom of second water absorber column 74. Stream 79, a first portion of the liquid raw methanol is injected in stream 107 to prevent hydrate formation in cryogenic temperatures of downstream equipment. Stream 78, a second portion of the liquid raw methanol is sent to a first liquid-liquid heat exchanger 116. Stream 80, a third portion of the liquid raw methanol is injected in stream 190 to prevent hydrate formation in cryogenic temperatures of downstream equipment.

The exhaust gases exit from the second water absorber column 74 through stream 75. The present invention provides systems and methods for separation methanol from gas using the second water absorber column 74 and water liquid. This configuration provides deep methanol removal. The second water absorber column 74 is necessary for downstream methanation reactors because methanol cracking at a high temperature can form coke, which reduces catalyst activity. A molar fraction of methanol in stream 75 is less than 1.0E-6.

The exhaust gas from the second water absorber column 74, stream 75, is mixed in a high pressure recycled carbon dioxide-rich gas stream 271 so that a mole percent of carbon dioxide content in the gas mixture of stream 81 exceeds 40.0%. Subsequently, the gas mixture of stream 81 is directed through heat exchangers 82 and 84, reaching a final temperature of approximately 250.0° C. and then they enter a first methanation reactor 86. Carbon monoxide and carbon dioxide gases are converted into methane during the sabatier reaction on the catalytic bed of the first methanation reactor 86.

CO2+4H2→CH4+2H2O

CO+3H2→CH4+H2O

Hot gas leaves the first methanation reactor 86 through stream 87 at a temperature of 650.0° C. Excess carbon dioxide gas absorbs the produced heat in the first methanation reactor 86. The hot gases from the first methanation reactor 86 enter the heat exchanger 88, which can superheat other vapors or partially or fully replace the furnace 42 for heating the feed of the RWGS reactor 44. The exhaust gases from the heat exchanger 88 enter a heat exchanger 90 to reduce their temperature to 250.0° C. by converting water liquid into steam and then they enter a second methanation reactor 92. Carbon monoxide and carbon dioxide gases are converted into methane during the sabatier reaction on the catalytic bed of the second methanation reactor 92.

CO2+4H2→CH4+2H2O

CO+3H2→CH4+H2O

Hot gas leaves the second methanation reactor 92 through stream 93 at a temperature of 650.0° C. Excess carbon dioxide gas absorbs the produced heat in the second methanation reactor 92. High-temperature methanation catalysts SNG-5000, produced by Clariant company, can be used in the methanation reactors 86 and 92. This methanation catalyst contains 53.0-60.0% (mass) NiO material. The hot gases from the second methanation reactor 92 enter the heat exchanger 94, which can superheat other vapors or partially or fully replace the furnace 42 for heating the feed of the RWGS reactor 44.

The exhaust gases from the heat exchanger 94 enter a heat exchanger 96 to reduce their temperature to 250.0° C. by converting water liquid into steam. After exiting the heat exchanger 96, the hot gases are passed through a water-cooled heat exchanger 98, where they are cooled to a temperature of 45.0° C. and then they enter a drum 100. It is possible to implement an air cooler before heat exchanger 98 (not shown for simplicity of the figure). Within the drum 100, water liquid is separated from the remaining gases. The water liquid from the drum 100 is sent to the second water absorber column 74 through stream 102, a pump 103 and stream 104 to use its water liquid to facilitate methanol absorption.

The exhaust gases from drum 100 enter a carbon dioxide recovery membrane unit 105, where they undergo a membrane separation process by DDR-type zeolite membranes. This process facilitates the separation of carbon dioxide from hydrogen, helium, nitrogen, methane, carbon monoxide and other gases. DDR-type zeolite membranes from NGK insulators company could be used for the carbon dioxide recovery membrane unit 105. Separated carbon dioxide gas is sent to the first water absorber column 250 through stream 106. Hydrogen, helium, nitrogen, methane, carbon monoxide and other gases are sent to a first coldbox 109 through streams 107 and 108. Stream 79, the first portion of the liquid raw methanol is injected in stream 107 to prevent hydrate formation in cryogenic temperatures of downstream equipment.

The gases of streams 108 are cooled in the first coldbox 109. The obtained gas and liquid from the first coldbox 109 are sent to a second reboiler of a propane distillation tower 111 through stream 110 and then they are sent to a first reboiler of a nitrogen stripper tower 113 through stream 112 and after that they are sent to a bottom of first carbon dioxide absorber tower 115 through stream 114, where the obtained gas is washed by a cryogenic temperature liquid methanol stream 119 and a first portion of a recycled cryogenic temperature liquid methanol stream 135 on the packed beds for water and carbon dioxide removal. Stream 78, the second portion of the liquid raw methanol is sent to the first liquid-liquid heat exchanger 116 and then it is cooled in a second liquid-liquid heat exchanger 118 to produce the cryogenic temperature liquid methanol stream 119 with a final temperature of −50.0° C. Due to TEGDE freezing point temperature, it is possible that a liquid-liquid separator to be installed between the first liquid-liquid heat exchanger 116 and the second liquid-liquid heat exchanger 118 to remove remaining TEGDE from methanol at low temperature. Vapor pressure of water and carbon dioxide in the outlet gas of the first carbon dioxide absorber tower 115 shall be adjusted to prevent solid water and solid carbon dioxide in downstream equipment.

The outlet gas of the first carbon dioxide absorber tower 115 is sent to a second reboiler of the nitrogen stripper tower 137 and then it is sent to a second coldbox 139 to cool by a liquid nitrogen refrigerant stream 173 and other cold streams. After reaching a final temperature of approximately −165.0° C., it is sent to a drum 141 to separate gaseous hydrogen, nitrogen, methane, carbon monoxide, and helium from liquid methane and other liquids. The liquid methane and other liquids from the bottom of drum 141 are sent to a second condenser of the nitrogen stripper tower 144, the second coldbox 139, and the first coldbox 109 (through stream 146) to warm up and then they mix in a heated non-condensable off-gas stream 176 from a reflux drum of the nitrogen stripper tower 158 (through stream 159) and a heated waste gas stream 232 from a reflux drum of the propane distillation tower 227 (through stream 228). This mixture, a methane-rich fuel product, is sent to petrochemical plants' fuel gas network through stream 177. The gaseous hydrogen, nitrogen, methane, carbon monoxide, and helium are sent to a first methane absorber tower 147 through stream 142 where nitrogen and carbon monoxide are separated from hydrogen and helium by a first portion of a recycled high-purity liquid methane stream 172. Temperature of stream 172 is less than −165.0° C. and its flowrate is selected to absorb nitrogen of streams 142 more than 99.9% (mole percent). The liquid nitrogen refrigerant stream 173, vaporizes in the second coldbox 139 and cools down hot streams and then vaporized nitrogen is sent to the first coldbox 109 through stream 174 to warm up and vent to a safe location through stream 178.

The gaseous hydrogen, helium, methane and a few other gases exit from the top of first methane absorber tower 147 and then they are sent to the second coldbox 139 through stream 149 and the first coldbox 109 through stream 150 to warm up and after that they are sent to a compressor 180 through stream 179. The compressed gaseous are heated in a heat exchanger 182 and then they are fed into a hydrogen catalytic combustion reactor 184, where a high-purity gaseous oxygen from stream 185 oxidize hydrogen and methane on a catalytic bed, according to the following reaction:

2H2+O2→2H2O

CH4+2O2→CO2+2H2O

CH4+O2→CO+2H2O

An excess oxygen gas through stream 185 and a catalyst type for the hydrogen catalytic combustion bed are selected in such way that a molar fraction of hydrogen in stream 185 is less than 5.0E-7. There is nitrogen in the outlet gases of hydrogen catalytic combustion reactor 184 because the high-purity gaseous oxygen from stream 185 has nitrogen impurity.

The outlet hot gases of hydrogen catalytic combustion reactor 184 are cooled in a heat exchanger 187 and then they enter a drum 189 where they are separated into liquids and gases. The liquid from the drum 189, which contains water liquid and some dissolved gases, is pumped to the second water absorber column 74 through streams 191, 193 and a pump 192 to use its water liquid for methanol absorption. Through stream 190, the exit gas from the drum 189, which contains helium, methane, water, carbon dioxide, carbon monoxide, nitrogen, oxygen, and some other impurities is mixed in the third portion of the liquid raw methanol stream 80, to prevent hydrate formation in cryogenic temperatures of downstream equipment. The mixture is sent to a first reboiler of the propane distillation tower 195 through stream 194 and after that it enters a second carbon dioxide absorber tower 196.

A second portion of the recycled cryogenic temperature liquid methanol stream 136 is sent to the top of second carbon dioxide absorber tower 196 for water and carbon dioxide removal. A cryogenic temperature liquid methanol-rich stream 197 is sent from the bottom of second carbon dioxide absorber tower 196 to the bottom of first carbon dioxide absorber tower 115. Vapor pressure of water and carbon dioxide in the outlet gas of the second carbon dioxide absorber tower 196 shall be adjusted to prevent solid water and solid carbon dioxide in downstream equipment. The outlet gas from the top of second carbon dioxide absorber tower 196 is send to a liquid propane heat exchanger 199 and after that it is sent to a second methane absorber tower 201 for nitrogen, oxygen, and carbon monoxide removal. A second portion of the recycled high-purity liquid methane stream 171 is routed from the second coldbox 139 to the top of second methane absorber tower 201 where nitrogen, oxygen, and carbon monoxide are separated from helium. Temperature of stream 171 is less than −165.0° C. and its flowrate is selected to reduce molar fraction of nitrogen, oxygen, and carbon monoxide in stream 203 to less than 1.0E-6.

The liquid stream 202 from the bottom of second methane absorber tower 201 is sent to the top of nitrogen stripper tower 153. The second methane absorber tower 201 outlet gases are sent to a propane absorber tower 204. A recycled high-purity liquid propane stream 224 is routed from a second condenser of the propane distillation tower 223 to the top of propane absorber tower 204 where methane is separated from helium. The outlet gas from the top of propane absorber tower 204 is send to activated carbon beds 207 through stream 206. Temperature of stream 224 is less than −150.0° C. and its flowrate is selected to reduce molar fraction of methane in stream 206 to less than 1.0E-6. A cryogenic temperature liquid propane-rich stream 205 is sent from a bottom of propane absorber tower 204 to the liquid propane heat exchanger 199 and after that it is sent to a first condenser of the propane distillation tower 211 and then it is sent to the propane distillation tower 213 where methane is stripped from liquid propane on the packed beds or trays to produce a high-purity liquid propane stream 215 from the bottom of propane distillation tower 213.

The propane distillation tower 213 overhead gases are sent to the first condenser of the propane distillation tower 211 and the second condenser of the propane distillation tower 223. The second condenser of the propane distillation tower 223 outlet gas and liquid are send to the reflux drum of the propane distillation tower 227 through stream 226 where they are separated into liquids and the waste gas. Temperature of stream 226 is less than −145.0° C. to prevent loss of propane. The liquid from the reflux drum of the propane distillation tower 227 is sent back to the top of the propane distillation tower 213 through a pump 229. is send to the first coldbox 109 through stream 228 to warm up and then it mix in the other gaseous methane source. This mixture, the methane-rich fuel product, is sent to petrochemical plants' fuel gas network through stream 177. If the propane distillation tower 213 work at a approximately atmospheric pressure, it is possible to use a compressor to increase pressure of the waste gas (not shown for simplicity of the figure).

A first portion of the high-purity liquid propane stream 217 from the bottom of propane distillation tower 213 is sent to the first reboiler of the propane distillation tower 195 to adjust methane impurity in the high-purity liquid propane from the bottom of propane distillation tower 213. Also a second portion of the high-purity liquid propane stream 218 from the bottom of propane distillation tower 213 is sent to the second reboiler of the propane distillation tower 111 to adjust methane impurity in the high-purity liquid propane from the bottom of propane distillation tower 213. A third portion of the high-purity liquid propane stream 219 from the bottom of propane distillation tower 213 is pumped to the second coldbox 139 to cool down through a pump 220 and stream 221 and then it is send to the second condenser of the propane distillation tower 223 to produce the recycled high-purity liquid propane stream 224. In next step, the recycled high-purity liquid propane stream 224 is routed from the second condenser of the propane distillation tower 223 to the top of propane absorber tower 204. A make-up high-purity liquid propane stream 216 is injected in the top of propane distillation tower 213 to compensate for gaseous propane in the methane-rich fuel product stream 177 and other losses through stream 206.

The gases exiting the top of propane absorber tower 204 enter the activated carbon beds 207 through stream 206. Cryogenic adsorption on the activated carbon beds 207 separates methane, nitrogen and other unwanted gases from helium. Stream 209 is a high-purity helium gas product. A mole percent of helium in stream 209 is greater than 99.999%. A ratio of a helium content by mole in stream 209 to a helium content by mole in stream 29 is greater than 0.71. The activated carbon adsorption beds 207 are designed with at least two parallel trains, each with a 100.0% capacity, so that one train is on stream for processing while the other undergoes regeneration. A portion of stream 206 could be heated and used as a regeneration gas. During the regeneration process, the adsorbed waste materials by the activated carbon beds are released as a tail gas and send to the bottom of first water absorber column 250 through stream 208 for helium loss prevention.

The cryogenic temperature liquid methanol stream 119 and the first portion of the recycled cryogenic temperature liquid methanol stream 135 absorb water and carbon dioxide in the first carbon dioxide absorber tower 115 and then a first carbon dioxide absorber tower outlet liquid methanol stream 121 exit from the bottom of first carbon dioxide absorber tower 115. After heating in the first liquid-liquid heat exchanger 116, a second portion of first carbon dioxide absorber tower outlet liquid methanol stream 125 is sent to a degassing drum 127 where it is separated into liquids and gases. The degassing drum 127 vent gas is sent to the first water absorber column 250 through stream 128. The degassing drum outlet liquid methanol, a methanol-water liquid mixture, is sent to a methanol plant is located in the outside of the battery limit for methanol purification, through stream 129. A first portion of first carbon dioxide absorber tower outlet liquid methanol stream 122 is pumped by a pump 123 to the first coldbox 109 through stream 124 to warm up and then it is sent to a drum 131 where it is separated into liquids and gases. The obtained gases from the drum 131 are sent to the degassing drum 127. Through stream 133, the liquid from the drum 131 is sent back to the first coldbox 109 to cool down and to produce a recycled cryogenic temperature liquid methanol stream 134 with a final temperature of −50.0° C. The first portion of the recycled cryogenic temperature liquid methanol stream 135 is sent to the top of first carbon dioxide absorber tower 115 for water and carbon dioxide removal. The second portion of the recycled cryogenic temperature liquid methanol stream 136 is sent to the second carbon dioxide absorber tower 196.

The liquid from the bottom of first methane absorber tower 147 is sent to a first condenser of the nitrogen stripper tower 151 and then it is sent to the top of nitrogen stripper tower 153 where nitrogen, oxygen, and carbon monoxide are stripped from liquid methane on the packed beds or trays to produce a high-purity liquid methane stream 154 from the bottom of nitrogen stripper tower 153. A first portion of the high-purity liquid methane stream 161 from the bottom of nitrogen stripper tower 153 is sent to the first reboiler of the nitrogen stripper tower 113 to adjust nitrogen impurity and oxygen impurity in the high-purity liquid methane stream 154 from the bottom of nitrogen stripper tower 153. Also a second portion of the high-purity liquid methane stream 163 from the bottom of nitrogen stripper tower 153 is sent to the second reboiler of the nitrogen stripper tower 137 to adjust nitrogen impurity and oxygen impurity in the high-purity liquid methane stream 154 from the bottom of nitrogen stripper tower 153.

A third portion of the high-purity liquid methane stream 165 from the bottom of nitrogen stripper tower 153 is pumped to the second coldbox 139 to cool down through a pump 166 and stream 167 and then it is send to the second liquid-liquid heat exchanger 118 through stream 168. In next step, it is sent to the second coldbox 139 from the second liquid-liquid heat exchanger 118 through stream 169 to produce a recycled high-purity liquid methane stream 170. After that, the first portion of the recycled high-purity liquid methane stream 172 is routed from the second coldbox 139 to the top of first methane absorber tower 147.

Through stream 155, overhead gases of the nitrogen stripper tower 153 are sent to the first condenser of the nitrogen stripper tower 151 and after that they are sent to the second condenser of the nitrogen stripper tower 144. Through stream 157, the outlet gas and liquid of second condenser of the nitrogen stripper tower 144 are send to the reflux drum of the nitrogen stripper tower 158 where they are separated into liquids and the non-condensable off-gas. The liquid from the reflux drum of the nitrogen stripper tower 158 is sent back to the nitrogen stripper tower 153 through a pump 160. The non-condensable off-gas stream 159 is send to the first coldbox 109 to warm up and then it mix in the other gaseous methane source. This mixture, the methane-rich fuel product, is sent to petrochemical plants' fuel gas network through stream 177.

The methanol-rich solvent is sent to the methanol stripper tower 233 from the TEGDE absorber column 61 through stream 62. In the methanol stripper tower 233, the absorbed materials by TEGDE are desorbed under low pressure conditions with the aid of a reboilers of the methanol stripper tower 237. The methanol-lean solvent exits the bottom of methanol stripper tower 233 via stream 235. Then, the methanol-lean solvent pressure is increased by a pump 239 and it is cooled by a water-cooled heat exchanger 241, achieving a final temperature of 45.0° C. It is possible to implement an air cooler before the water-cooled heat exchanger 241 (not shown for simplicity of the figure). After that, the methanol-lean solvent is sent to the top of TEGDE absorber column 61 through stream 242.

To prevent TEGDE loss, a stripped gases from the top of methanol stripper tower 233 are cooled less than 75.0° C. in a partial condenser 243. The liquids and gases from the partial condenser 243 is sent to a drum 245 for phase separation. The liquid phase from the drum 245 is recycled to the methanol stripper tower 233 via stream 246, while the gas phase is vented via stream 247. the vent gas from the drum 245 is sent to a water-cooled heat exchanger 248, achieving a final temperature of 45.0° C., and then it is fed to the bottom of first water absorber column 250 through stream 249 to capture methanol. Within the first water absorber column 250, methanol is absorbed by water liquid entering from the top of first water absorber column 250 through stream 281 on the packed bed. Stream 281 contains liquid demineralized water. Subsequently, a liquid mixture of water and methanol exit the bottom of first water absorber column 250 through stream 252 and it is sent to the bottom of second water absorber column 74 via pump 253 and stream 254. A carbon dioxide-rich feedstock gas stream 280 enters the bottom of first water absorber column 250.

Through stream 251, a overhead gases of the top of first water absorber column 250 are sent to series-connected compressors 255, 262, 269, and 278 to increase their pressure. Between all of the compressors, the gases are cooled down, using exchangers 257, 264, and 273. Subsequently, the cooled gases undergo liquid phase and gas phase separation in drums 259, 266, and 275. A first portion of the compressed overhead gases of the first water absorber column, the high-high pressure recycled carbon dioxide-rich gas stream 279, is combined with stream 31, so that the molar ratio of hydrogen to carbon dioxide ranges 1.0 to 8.0 in stream 35. A second portion of the compressed overhead gases of the first water absorber column, the high pressure recycled carbon dioxide-rich gas stream 271, are mixed in stream 75 so that a mole percent of carbon dioxide content in the gas mixture of stream 81 exceeds 40.0%. A molar fraction of methanol in stream 251 is less than 1.0E-6. The liquid phase from the drum 275 is sent to the drum 259 via stream 276 and the liquid phase from the drums 259 and 266 is sent to the top of first water absorber column 250 through streams 260 and 267.

What is claimed is:

1. A method of obtaining a high-purity helium gas product with simultaneous production of a methanol-water liquid mixture and a methane-rich fuel product from a hydrogen-rich feedstock gas containing helium, a carbon dioxide-rich feedstock gas, and an inert methane-rich gas comprising:
(a) introducing the hydrogen-rich feedstock gas containing helium, a solvent recovery unit (SRU) high-high pressure recycled carbon dioxide-rich gas, and the inert methane-rich gas to a reverse water gas shift unit (RWGSU), being configured to generate carbon monoxide and water from carbon dioxide and hydrogen within a RWGSU reverse water gas shift reactor at a low pressure and in the presence of the inert methane-rich gas, to produce a RWGSU gaseous carbon monoxide and a RWGSU liquid water, wherein a molar ratio of carbon monoxide to hydrogen in the RWGSU gaseous carbon monoxide is greater than a molar ratio of carbon monoxide to hydrogen in the RWGSU inlet gases mixture;
(b) introducing the RWGSU gaseous carbon monoxide and a methanol-lean solvent from the solvent recovery unit (SRU) to a methanol production unit (MEP), being configured to generate methanol and water from hydrogen, carbon dioxide, and carbon monoxide, within series-connected methanol reactors and also being configured for selective absorption of the generated methanol and water between the reactors with a high-boiling inert solvent within solvent absorber columns, to produce a MEP gaseous methanol and a methanol-rich solvent, wherein a molar ratio of methanol in the MEP gaseous methanol is greater than a molar ratio of methanol in the RWGSU gaseous carbon monoxide, and wherein a molar ratio of methanol in the methanol-rich solvent is greater than a molar ratio of methanol in the methanol-lean solvent;
(c) introducing the MEP gaseous methanol, a SRU methanol-rich water liquid, a methane production unit (CH4P) liquid water, a catalytic oxidation unit (COXU) liquid water, and a first portion of a liquid demineralized water to a methanol absorption unit (MEA), being configured for methanol absorption with water liquid within a second water absorber column, to produce a MEA methanol-free gas and a MEA methanol-aqueous solution, wherein a molar fraction of methanol in the MEA methanol-free gas is less than 1.0E-6;
(d) introducing the MEA methanol-free gas and a SRU high pressure recycled carbon dioxide-rich gas to the methane production unit (CH4P), being configured to generate methane and water from hydrogen, carbon dioxide, and carbon monoxide within series-connected methanation reactors, in the presence of an excess carbon dioxide gas, to produce a CH4P gaseous methane and the CH4P liquid water, wherein a mole percent of carbon dioxide in the mixture of the MEA methanol-free gas and the SRU high pressure recycled carbon dioxide-rich gas exceeds the stoichiometric requirement, reaching up to 40.0%, and wherein a molar ratio of methane in the CH4P gaseous methane is greater than a molar ratio of methane in the CH4P inlet gases mixture;
(e) introducing the CH4P gaseous methane to a carbon dioxide recovery membrane unit (CRMU), being configured to selectively separate nitrogen, methane, hydrogen, helium, and other gases from carbon dioxide to produce a CRMU gaseous carbon dioxide and a CRMU gaseous methane, wherein a molar ratio of carbon dioxide in the CRMU gaseous carbon dioxide is more than a molar ratio of carbon dioxide in the CH4P gaseous methane;
(f) introducing the CRMU gaseous methane, the MEA methanol-aqueous solution, and a liquid nitrogen refrigerant to a first portion of a cryogenic nitrogen rejection unit (CNR), being configured for absorption of carbon dioxide and water with a cryogenic temperature liquid methanol within a first carbon dioxide absorber tower, and being configured for absorption of nitrogen and carbon monoxide with a liquid methane within a first methane absorber tower, to produce a CNR gaseous hydrogen, the methane-rich fuel product, the methanol-water liquid mixture, a CNR gaseous nitrogen vent gas, and a CNR degassing drum vent gas, wherein a molar ratio of nitrogen in the CNR gaseous hydrogen is less than a molar ratio of nitrogen in the CRMU gaseous methane;
(g) introducing the CNR gaseous hydrogen and an oxygen-rich gas to the catalytic oxidation unit (COXU), being configured to remove hydrogen and methane within a hydrogen catalytic combustion reactor, to produce a COXU gaseous helium and the COXU liquid water, wherein a molar fraction of hydrogen in the COXU gaseous helium is less than 1.0E-6;
(h) introducing the COXU gaseous helium and a high-purity liquid propane to a second portion of the cryogenic nitrogen rejection unit (CNR), being configured for absorption of carbon dioxide and water with a cryogenic temperature liquid methanol within a second carbon dioxide absorber tower, and being configured for absorption of nitrogen and carbon monoxide with a liquid methane within a second methane absorber tower, and being configured for absorption of methane with a cryogenic temperature liquid propane within a propane absorber tower, to produce a CNR gaseous helium, wherein a mole percent of helium in the CNR gaseous helium is greater than 99.99%;
(i) introducing the CNR gaseous helium to a cryogenic adsorption unit (CAU), being configured to adsorb methane, nitrogen, and other unwanted gases from helium on activated carbon beds at a cryogenic temperature, to produce the helium-rich gas product and a CAU tail gas, wherein a mole percent of helium in the high-purity helium gas product is greater than 99.999%; and
(j) introducing the RWGSU liquid water, the methanol-rich solvent, the CRMU gaseous carbon dioxide, the carbon dioxide-rich feedstock gas, the CNR degassing drum vent gas, the CAU tail gas, and a second portion of the liquid demineralized water to the solvent recovery unit (SRU), being configured to strip the absorbed methanol and water from the methanol-rich solvent within a methanol stripper tower, and being configured to adsorb the stripped methanol gas with water liquid streams within a first water absorber column, and being configured to increase pressure of nonabsorbable gases of SRU inlet streams, to produce the SRU high pressure recycled carbon dioxide-rich gas, the SRU high-high pressure recycled carbon dioxide-rich gas, the methanol-lean solvent, and the SRU methanol-rich water liquid, wherein the molar ratio of methanol in the methanol-rich solvent is greater than the molar ratio of methanol in the methanol-lean solvent, and wherein a molar fraction of methanol in the SRU gaseous carbon dioxide is less than 1.0E-6.

2. The method of claim 1, wherein a ratio of a helium content by mole in the high-purity helium gas product to a helium content by mole in the hydrogen-rich feedstock gas containing helium is greater than 0.71.

3. The method of claim 1, wherein a molar ratio of hydrogen to carbon dioxide ranges from 1.0 to 8.0 in the mixture of the hydrogen-rich feedstock gas containing helium, the first portion of the inert methane-rich gas, and the SRU high-high pressure recycled carbon dioxide-rich gas depends on the type of RWGSU catalysts, a operating temperature of the RWGSU reverse water gas shift reactor, and a operating pressure of the RWGSU reverse water gas shift reactor, and wherein a molar ratio of carbon dioxide to methane in the mixture of the hydrogen-rich feedstock gas containing helium, the inert methane-rich gas, and the SRU high-high pressure recycled carbon dioxide-rich gas ranges from 1.0 to 4.5, depends on the methane selectivity of the reverse water gas shift reaction, and wherein a mole percent of carbon dioxide in the mixture of the hydrogen-rich feedstock gas containing helium, the inert methane-rich gas, and the SRU high-high pressure recycled carbon dioxide-rich gas exceeds 10.0%, and wherein a mole percent of methane in the mixture of the hydrogen-rich feedstock gas containing helium, the first portion of the inert methane-rich gas, and the SRU high-high pressure recycled carbon dioxide-rich gas exceeds 10.0%.

4. The method of claim 1, wherein the reverse water gas shift unit (RWGSU) comprises at least and respectively a turboexpander, a fired heater, the RWGSU reverse water gas shift reactor, a RWGSU cooler, a RWGSU gas-liquid separator, and a RWGSU compressor.

5. The method of claim 1, wherein the methanol production unit (MEP) comprises at least and respectively a first methanol reactor, a solvent absorber column, a second methanol reactor, and a MEP cooler.

6. The method of claim 1, wherein the methanol absorption unit (MEA) comprises at least the second water absorber column.

7. The method of claim 1, wherein the methane production unit (CH4P) comprises at least and respectively a CH4P heat exchanger, a methanation reactor, a CH4P cooler, and a CH4P gas-liquid separator.

8. The method of claim 1, wherein the carbon dioxide recovery membrane unit (CRMU) comprises at least a DDR-type zeolite membrane.

9. The method of claim 1, wherein the first portion of the cryogenic nitrogen rejection unit (CNR) comprises at least a coldbox, a nitrogen stripper tower, the first carbon dioxide absorber tower, the first methane absorber tower.

10. The method of claim 1, wherein the catalytic oxidation unit (COXU) comprises at least and respectively a COXU heat exchanger, the hydrogen catalytic combustion reactor, a COXU cooler, and a COXU gas-liquid separator.

11. The method of claim 1, wherein the second portion of the cryogenic nitrogen rejection unit (CNR) comprises at least a propane distillation tower, the second carbon dioxide absorber tower, the second methane absorber tower, and the propane absorber tower.

12. The method of claim 1, wherein the cryogenic adsorption unit (CAU) comprises at least two activated carbon beds.

13. The method of claim 1, wherein the solvent recovery unit (SRU), comprises at least and respectively the methanol stripper tower, the first water absorber column, and a multi stage compressor with interstage coolers and separator drums.

14. A method of obtaining an elevated pressure carbon monoxide product and a cooled liquid water product from a hydrogen-rich feedstock gas, a carbon dioxide-rich feedstock gas, and an inert methane-rich gas comprising:
(a) the hydrogen-rich feedstock gas, the carbon dioxide-rich feedstock gas and the inert methane-rich gas are mixed, to produce a mixed feed gas, wherein a molar ratio of hydrogen to carbon dioxide in the mixed feed gas ranges from 1.0 to 8.0, depends on downstream reverse water gas shift reactor operating temperature, pressure and catalyst type, and wherein a molar ratio of carbon dioxide to methane in the mixed feed gas ranges from 1.0 to 4.5, depends on the methane selectivity of the reverse water gas shift reaction, and wherein a mole percent of carbon dioxide in the mixed feed gas exceeds 10.0%, and wherein a mole percent of methane in the mixed feed gas exceeds 10.0%;
(b) the mixed feed gas is heated to an outlet temperature greater than 90.0° C., to produce a warmed-up gas in an exchanger heater;
(c) the warmed-up gas is depressurized to produce a low pressure feed gas in a turboexpander;
(d) the low pressure feed gas is heated to an outlet temperature greater than 650.0° C., to produce a heated low pressure feed gas in a furnace;
(e) the heated low pressure feed gas is sent to a reverse water gas shift reactor;
(f) the reverse water gas shift reactor contains a catalyst that converts the heated low pressure feed gas to a product gas which contains carbon monoxide and water, wherein methane content of the heated low pressure feed gas prevent methanation reactions in the reverse water gas shift reactor;
(g) the product gas is cooled to an outlet temperature less than 45.0° C., to produce a cooled gas and the cooled liquid water product in an exchanger cooler;
(h) the cooled gas is sent to a water-gas separator to produce a dried product gas; and
(i) the dried product gas is pressurized, to produce the elevated pressure carbon monoxide product in a compressor, wherein the turboexpander works as a driver for the compressor.

15. A method of obtaining a methanol-water liquid mixture and an elevated pressure residual gas mixture from a hydrogen-rich feedstock gas which contains carbon monoxide, a carbon dioxide-rich feedstock gas, and an inert methane-rich gas comprising:
(a) the hydrogen-rich feedstock gas which contains carbon monoxide, the carbon dioxide-rich feedstock gas and the inert methane-rich gas are mixed, to produce a mixed feed gas, wherein a mole percent of carbon dioxide in the mixed feed gas exceeds 10.0%, and wherein a mole percent of methane in the mixed feed gas exceeds 10.0%;
(b) the mixed feed gas is heated to an outlet temperature greater than 230.0° C., to produce a heated feed gas in exchanger heaters;
(c) the heated feed gas is sent to a first methanol reactor;
(d) the first methanol reactor contains a catalyst that converts the heated feed gas to a raw methanol product gas which contains methanol, carbon dioxide, carbon monoxide, methane and water, wherein methane content of the heated feed gas prevent methanation reactions in the first methanol reactor, and wherein the carbon dioxide content within the heated feed gas facilitates methanol production;
(e) the raw methanol product gas is directed to a first absorption column employing a cooled methanol-lean solvent for the selective absorption of methanol and water, yielding a methanol-rich solvent and a methanol-free gas, wherein main component of the cooled methanol-lean solvent is a high-boiling inert solvent, such as tetraethylene glycol dimethyl ether (tetraglyme) (TEGDE);
(f) the methanol-free gas is sent to a second methanol reactor;
(g) the second methanol reactor contains a catalyst that converts the methanol-free gas to a methanol gas mixture which contains methanol, carbon dioxide, carbon monoxide, methane and water, wherein methane content of the methanol-free gas prevent methanation reactions in the second methanol reactor, and wherein the carbon dioxide content within the methanol-free gas facilitates methanol production;
(h) the methanol gas mixture is cooled to an outlet temperature less than 45.0° C., to produce a cooled methanol gas mixture in an exchanger cooler;
(i) the methanol-rich solvent is directed to a methanol stripper tower, yielding a methanol-lean solvent and a stripped methanol gas;
(j) the methanol-lean solvent is cooled to an outlet temperature less than 45.0° C., to produce the cooled methanol-lean solvent in an exchanger cooler;
(k) the cooled methanol-lean solvent is routed to the first absorption column through a pump;
(l) the stripped methanol gas is cooled to an outlet temperature less than 45.0° C., to produce a cooled stripped methanol gas in an exchanger cooler;
(m) the cooled methanol gas mixture and the cooled stripped methanol gas are directed to a second absorption column employing water liquid for the selective absorption of methanol, yielding the methanol-water liquid mixture and a residual gas mixture composed of methane and carbon dioxide; and
(n) the residual gas mixture is directed to a compressor to produce an elevated pressure residual gas mixture.
